SEQUENCE LISTING (6 sequences)

SEQ ID NO: 1 - DNA, 112 bp, Artificial Sequence
  Cut 1 target specific sequence and scaffold sequence
ctcgactgccggcgacggccgtttaagagctatgctggaacagcatagcaagtttaaataaggctagtccgttatcaacttgaaaaagtggcaccgagtcggtgcttttttt

SEQ ID NO: 2 - DNA, 113 bp, Artificial Sequence
  Cut 2 target specific sequence and scaffold sequence
gaggccatcgcccgtcccttgtttaagagctatgctggaaacagcatagcaagtttaaataaggctagtccgttatcaacttgaaaaagtggcaccgagtcggtgcttttttt

SEQ ID NO: 3 - DNA, 113 bp, Artificial Sequence
  Bind 1 target specific sequence and scaffold sequence
gctttttgatccttcgatgtgtttaagagctatgctggaaacagcatagcaagtttaaataaggctagtccgttatcaacttgaaaaagtggcaccgagtcggtgcttttttt

SEQ ID NO: 4 - DNA, 113 bp, Artificial Sequence
  Bind 2 target specific sequence and scaffold sequence
tccgcaccggaccccggtccgtttaagagctatgctggaaacagcatagcaagtttaaataaggctagtccgttatcaacttgaaaaagtggcaccgagtcggtgcttttttt

SEQ ID NO: 5 - DNA, 112 bp, Artificial Sequence
  Bind 3 target specific sequence and scaffold sequence
ggagcccgccccctccgggggtttaagagctatgctggaacagcatagcaagtttaaataaggctagtccgttatcaacttgaaaaagtggcaccgagtcggtgcttttttt

SEQ ID NO: 6 - DNA, 113 bp, Artificial Sequence
  Bind 4 target specific sequence and scaffold sequence
aaccaggattccctcagtaagtttaagagctatgctggaaacagcatagcaagtttaaataaggctagtccgttatcaacttgaaaaagtggcaccgagtcggtgcttttttt

(19) United States
(12) Patent Application Publication
(10) Pub. No.: US 2023/0235393 A1
(43) Pub. Date: Jul. 27, 2023

(54) METHODS OF ENRICHING FOR TARGET NUCLEIC ACID MOLECULES AND USES THEREOF

(71) Applicant: QIAGEN Sciences, LLC, Germantown, MD (US)

(72) Inventors: Yexun WANG, Ellicott City, MD (US); Quan PENG, Clarksburg, MD (US); Nathan BLEWETT, Frederick, MD (US)

(21) Appl. No.: 18/001,329

(22) PCT Filed: Jun. 11, 2021
(86) PCT No.: PCT/US2021/036966

§ 371 Date: Dec. 9, 2022

Related U.S. Application Data
(60) Provisional application No. 63/038,507, filed on Jun. 12, 2020.

(51) Int. Cl. C12Q 1/6874

(52) U.S. Cl. CPC C12Q 1/6874; C12Q 1/6818

(57) ABSTRACT

The invention relates to methods of enriching for target nucleic acid molecules, More particularly, the methods of enriching for target nucleic acid molecules comprise binding target nucleic acid molecules in a sample with one or more first target endonucleases that are specific to a first locus of a target region of the target nucleic acid molecules, separating the target nucleic acid molecules from nontarget nucleic acid molecules in the sample, and binding the separated target nucleic acid molecules with one or more second target endonucleases that are specific to a second locus of the target region of the target nucleic acid molecules, and uses thereof.

### REFERENCE TO SEQUENCE LISTING SUBMITTED ELECTRONICALLY

The content of the electronically submitted sequence listing in ASCII text file (Name: 2495-0015WO01_Sequence Listing_ST25.txt; Size: 3 KB; and Date of Creation: Jun. 11, 2021) filed with the application is incorporated herein by reference in its entirety.

### FIELD OF THE INVENTION

The invention relates to methods of enriching for target nucleic acid molecules. More particularly, the methods of enriching for target nucleic acid molecules comprise binding target nucleic acid molecules in a sample with one or more first target endonucleases that are specific to a first locus of a target region of the target nucleic acid molecules and binding the target nucleic acid molecules with one or more second target endonucleases that are specific to a second locus of the target region of the target nucleic acid molecules, and uses thereof.

### BACKGROUND OF THE INVENTION

Next-generation sequencing and third generation single molecule sequencing technology has been used for nucleic acid analysis, e.g., in DNA variant detection as well as in RNA transcriptome profiling. In many applications, to reduce sequencing cost and/or achieve deeper coverage, sequencing targeted regions is preferred over sequencing the whole genome. Various approaches have been used to enrich sub-regions of genome before sequencing. For example, target specific hybridization probes can be used to pull down target regions from a whole genome library. Target specific PCR primers can also be used to amplify specific regions for sequencing.

CRISPR/Cas system is a sequence specific endonuclease system. Functional CRISPR system consists of various Cas protein and CRISPR RNA (crRNA). The sequence specificity of CRISPR/Cas system is provided by the combination of PAM (protospacer adjacent motif) specific for different Cas proteins and crRNA complementary to the target sequence immediately after PAM sequence. Various types of Cas proteins have been identified. The most commonly used Cas9 protein requires both crRNA and additional trans-activating CRISPR RNA (tracrRNA) to be functional. To simplify the use, crRNA and tracrRNA can be fused into one “single guide RNA” (sgRNA) and form a functional complex with Cas9. Other Cas proteins, e.g., Cas12a, only require crRNA to form a functional complex.

In addition to its wide use in in vivo gene editing work, CRISPR/Cas system has also been used in in vitro DNA/RNA manipulations, e.g., in DNA/RNA sequence enrichment. Compared to other enrichment methods, CRISPR/Cas based enrichment has several advantages. Cas protein/gRNA binding to target sequence happens at physiological temperature and at much faster speed than oligo probe hybridization, so the handling is much easier than probe capture enrichment. CRISPR/Cas system can be used in enrichment without PCR amplification, so native DNA/RNA modification can be preserved and directly read in Nanopore sequencing. CRISPR/Cas system also looks very attractive in terms of enriching very long DNA fragments, which has been difficult using traditional hybridization and amplification approach.

Recently, CRISPR/Cas system has also been demonstrated as a viable approach for target enrichment. Target regions can be either cut out from the rest of the genome by active Cas9-guide RNA complex or pulled down by inactive dCas9-guide RNA complex. Comparing to probe hybridization capture and PCR enrichment, CRISPR/Cas enrichment process can be much faster and does not require high temperature or cycling conditions. Although in theory, binding of Cas protein/gRNA complex to target DNA should be sequence specific, all reported CRISPR/Cas based enrichment examples showed low enrichment specificity, e.g., <10%. It appears that the Cas/gRNA complex-target interaction may not be so specific or other nonspecific process retains many nontarget DNA in the enrichment step.

To improve the efficiency of CRISPR/Cas based enrichment and sequencing, current fold of enrichment needs to be improved.

### SUMMARY OF THE INVENTION

Disclosed herein are methods of enriching for target nucleic acid molecules, comprising (a) binding target nucleic acid molecules in a sample with a first Cas protein/gRNA complex that is specific to a first locus of a target region of the target nucleic acid molecules; (b) separating the target nucleic acid molecules of (a) from nontarget nucleic acid molecules in the sample; and (c) binding the separated target nucleic acid molecules of (b) with a second Cas protein/gRNA complex that is specific to a second locus of the target region of the target nucleic acid molecules.

In some embodiments, the ends of the target and/or nontarget nucleic acid molecules in the sample are blocked by dephosphorylation, attaching a hairpin oligonucleotide, or nucleotide addition before the binding with the first and/or the second Cas protein/gRNA complex.

The methods disclosed herein can further comprise separating the target nucleic acid molecules of (c) from nontarget nucleic acid molecules.

In some embodiments, the first locus is different from the second locus.

In some embodiments, the first Cas protein/gRNA complex comprises an active Cas protein and the second Cas protein/gRNA complex comprises an active Cas protein. In some embodiments, the first Cas protein/gRNA complex comprises an active Cas protein and the second Cas protein/gRNA complex comprises an inactive Cas protein. In other embodiments, the first Cas protein/gRNA complex comprises an inactive Cas protein and the second Cas protein/gRNA complex comprises an active Cas protein. In some embodiments, the first Cas protein/gRNA complex comprises an inactive Cas protein and the second Cas protein/gRNA complex comprises an inactive Cas protein.

The active Cas protein can cut the target nucleic acid molecules.

The methods disclosed herein can further comprise ligating an adapter oligonucleotide to the cut ends of the target nucleic acid molecules. In some embodiments, the adapter oligonucleotide, the Cas protein, the gRNA, or the target nucleic acid molecules are attached to an affinity label.

In some embodiments, the methods further comprise attaching an affinity label to the adapter oligonucleotide, the Cas protein, the gRNA, or the target nucleic acid molecules.

The affinity label can be an affinity tag for binding to a solid surface, His tag, TAP tag, or antibody.

In some embodiments, the separating is performed by binding the target nucleic acid molecules bound to the affinity label to an affinity label partner and eluting the bound target nucleic acid molecules.

The adapter oligonucleotide ligated to the target nucleic acid molecules cut by the first Cas protein/gRNA complex can be attached to an affinity label.

The methods can further comprise eluting the target nucleic acid molecules bound to the affinity label to an affinity label partner before the binding in (c).

In some embodiments, the inactive Cas protein or gRNA is attached to an affinity label.

The methods can further comprise binding the inactive Cas protein or gRNA attached to an affinity label to an affinity label partner and eluting the inactive Cas protein bound target nucleic acid molecules.

In some embodiments, the affinity label is an anti-dCas antibody linked to a bead.

The first Cas protein/gRNA complex can comprise a set of active Cas protein/gRNA complexes or inactive Cas protein/gRNA complexes that are specific to a set of first loci of 2 or more different target regions.

The second Cas protein/gRNA complex can comprise a set of active Cas protein/gRNA complexes or inactive Cas protein/gRNA complexes that are specific to a set of second loci of 2 or more different target regions.

The first Cas protein/gRNA complex can comprise a set of inactive Cas protein/gRNA complexes that are specific to a set of first loci of 2 or more different target regions.

The second Cas protein/gRNA complex can comprise a set of inactive Cas protein/gRNA complexes that are specific to a set of second loci of 2 or more different target regions.

Also disclosed herein are methods of enriching for target nucleic acid molecules, comprising (a) binding target nucleic acid molecules in a sample with a Cas protein/gRNA complex specific to a first locus of a target region of the target nucleic acid molecules; (b) binding target nucleic acid molecules in the sample with a Cas protein/gRNA complex specific to a second locus of a target region of the target nucleic acid molecules; and (c) separating the target nucleic acid molecules from nontarget nucleic acid molecules in the sample.

In some embodiments, the ends of the target and/or nontarget nucleic acid molecules in the sample are blocked by dephosphorylation, attaching a hairpin oligonucleotide, or nucleotide addition before the binding with the Cas protein/gRNA complex in (a) and/or (b).

In some embodiments, the first locus of the target region is bound by a Cas protein/gRNA complex comprising an active Cas protein and the second locus of the target region is bound by a Cas protein/gRNA complex comprising an inactive Cas protein. The first locus of the target region can be bound by a Cas protein/gRNA complex comprising an inactive Cas protein and the second locus of the target region can be bound by a Cas protein/gRNA complex comprising an active Cas protein. The active and inactive Cas protein/gRNA complexes can bind to the target nucleic acid molecules in a same reaction.

The active Cas protein/gRNA complex can cut the target nucleic acid molecules.

The methods can further comprise ligating an adapter oligonucleotide to cut ends of the target nucleic acid molecules. In some embodiments, the adapter oligonucleotide, the Cas protein, the gRNA, or the target nucleic acid molecules are attached to an affinity label. In some embodiments, the methods further comprise attaching an affinity label to the adapter oligonucleotide, the Cas protein, the gRNA, or the target nucleic acid molecules. The affinity label can be an affinity tag for binding to a solid surface, His tag, TAP tag, or antibody.

In some embodiments, the separating is performed by binding the target nucleic acid molecules bound to the affinity label to an affinity label partner and eluting the bound target nucleic acid molecules.

In some embodiments, the first Cas protein/gRNA complex comprises a set of active Cas protein/gRNA complexes or inactive Cas protein/gRNA complexes that are specific to a set of first loci of 2 or more different target regions. In some embodiments, the second Cas protein/gRNA complex comprises a set of active Cas protein/gRNA complexes or inactive Cas protein/gRNA complexes that are specific to a set of second loci of 2 or more different target regions.

Further disclosed herein are methods of enriching for target nucleic acid molecules, comprising: (a) binding target nucleic acid molecules in a sample with one or more first target endonucleases that are specific to a first locus of a target region of the target nucleic acid molecules; (b) separating the target nucleic acid molecules from nontarget nucleic acid molecules in the sample; and (c) binding the separated target nucleic acid molecules with one or more second target endonucleases that are specific to a second locus of the target region of the target nucleic acid molecules.

The ends of the target and/or nontarget nucleic acid molecules in the sample can be blocked by dephosphorylation, attaching a hairpin oligonucleotide, or nucleotide addition before the binding with the first and/or the second target endonucleases.

The methods can further comprise separating the target nucleic acid molecules of (c) from nontarget nucleic acid molecules.

The methods can further comprise binding the separated target nucleic acid molecules with one or more third target endonucleases that are specific to a third locus of the target region and separating the target nucleic acid molecules from nontarget nucleic acid molecules.

The first target endonucleases and the second target endonucleases can target different loci of the target region. The first target endonucleases, the second target endonucleases, and the third target endonucleases can target different loci of the target region. The methods can enrich for multiple target regions.

The methods can further comprise releasing the target nucleic acid molecules from the first or second target endonucleases.

The one or more target endonucleases can be an active or inactive Cas protein, a Cas9-like enzyme, a Cpfl enzyme, a ribonucleoprotein, a meganuclease, a transcription activator-like effector-based nuclease (TALEN), a zinc-finger nuclease, an argonaute nuclease, a megaTAL nuclease, or a combination thereof. The one or more target endonucleases can be Cas9, CPFl, or a derivative thereof. The target endonucleases can be active or inactive Cas enzyme or Cpfl enzyme.

The binding can comprise cutting the nucleic acid molecules with the target endonucleases.

The target endonucleases can comprise a set of Cas enzymes that bind to 2 or more different loci in the same target region or different target regions. The Cas enzymes can comprise the same type of Cas enzyme. The Cas enzymes can comprise two or more different types of Cas enzymes.

In some embodiments, the separating the target nucleic acid molecules from the nontarget nucleic acid molecules can comprise gel electrophoresis, gel purification, liquid chromatography, size exclusion purification, filtration, SPRI bead purification, or enzymatic digestion of the nontarget nucleic acid molecules.

The methods can further comprise ligating an adapter to at least one of the 5′ or 3′ ends of the cut target nucleic acid molecules.

A transposase can be tethered to the first or second target endonuclease and the tethered transposase inserts a transposon end sequence tag in or near the binding site of the endonuclease. The transposase can be tethered to the target endonuclease through protein fusion.

The one or more target endonucleases can remain bound to the target region of the target nucleic acid molecules.

At least one target endonuclease or adapter can be attached to an affinity label. The affinity label is or comprises at least one of Acrydite, azide, azide (NHS ester), digoxigenin (NHS ester), Thinker, Amino modifier C6, Amino modifier C12, Amino modifier C6 dT, Unilink amino modifier, hexynyl, 5-octadiynyl dU, biotin, biotin (azide), biotin dT, biotin TEG, dual biotin, PC biotin, desthiobiotin TEG, thiol modifier C3, dithiol, thiol modifier C6 S-S, and/or succinyl groups.

The affinity label can be an affinity tag for binding to a solid surface, His tag, TAP tag, or antibody.

The methods can further comprise capturing the target nucleic acid molecules with an affinity label partner. The affinity label partner is or comprises at least one of amino silane, epoxy silane, isothiocyanate, aminophenyl silane, aminpropyl silane, mercapto silane, aldehyde, epoxide, phosphonate, streptavidin, avidin, an antibody, a hapten recognizing an antibody, a particular nucleic acid sequence, magnetically attractable particles, and/or photolabile resins.

The methods can further comprise analyzing the target nucleic acid molecules. The analyzing can comprise quantitation and/or sequencing of the target region. The quantitation can comprise at least one of spectrophotometric analysis, real-time PCR, and/or fluorescence-based quantitation. The sequencing can comprise next-generation sequencing, third-generation sequencing, duplex sequencing, SPLiT-duplex sequencing, Sanger sequencing, shotgun sequencing, bridge amplification/sequencing, nanopore sequencing, single molecule real-time sequencing, ion torrent sequencing, pyrosequencing, digital sequencing, direct digital sequencing, sequencing by ligation, polony-based sequencing, electrical current-based sequencing, sequencing via mass spectroscopy, microfluidics-based sequencing, and combinations thereof.

### BRIEF DESCRIPTION OF THE FIGURES/DRAWINGS

FIG. 1. A combination of active and/or inactive CRISPR/Cas enrichment systems are used sequentially with a separation step in between.

FIG. 2. A combination of active CRISPR/Cas enrichment systems are used sequentially with a separation step in between, with two gRNA targeting the same target region at different sites.

FIG. 3. A combination of active and inactive CRISPR/Cas enrichment systems are used sequentially with a separation step in between, with two gRNA targeting the same target region at different sites.

FIG. 4. A combination of inactive and active CRISPR/Cas enrichment systems are used sequentially with a separation step in between, with two gRNA targeting the same target region at different sites.

FIG. 5. A combination of inactive CRISPR/Cas enrichment systems are used sequentially with a separation step in between, with two gRNA targeting the same target region at different sites.

FIG. 6. A combination of active and/or inactive CRISPR/Cas enrichment systems are applied together in the same reaction tube.

FIG. 7. A combination of active and inactive CRISPR/Cas enrichment systems are applied together in the same reaction tube.

FIG. 8. For a method using a combination of active and inactive CRISPR/Cas enrichment systems in the same reaction tube, 6 sgRNAs were designed to target human ribosomal gene for 28S rRNA. Four sgRNAs were assembled with inactive Cas9 (dCas9) for binding enrichment (Bind 1, 2, 3, 4). The other two were assembled with active Cas9 for cutting enrichment (Cut 1, 2). The target position and direction of these sgRNAs in relating to the repeating unit are indicated.

FIG. 9. A combination of Cas (or dCas)-transposase fusion and/or dCas protein enrichment systems are used sequentially with a separation step in between.

FIG. 10. A combination of Cas (or dCas)-transposase fusion and/or dCas protein enrichment systems are used sequentially with a separation step in between, with the Cas tethered tagmentation used in the first round.

FIG. 11. A combination of Cas (or dCas)-transposase fusion and/or dCas protein enrichment systems are used sequentially with a separation step in between, with the Cas tethered tagmentation used in the second round.

FIG. 12. A combination of Cas (or dCas)-transposase fusion and/or dCas protein enrichment systems are used sequentially with a separation step in between, with the Cas tethered tagmentation used in both rounds.

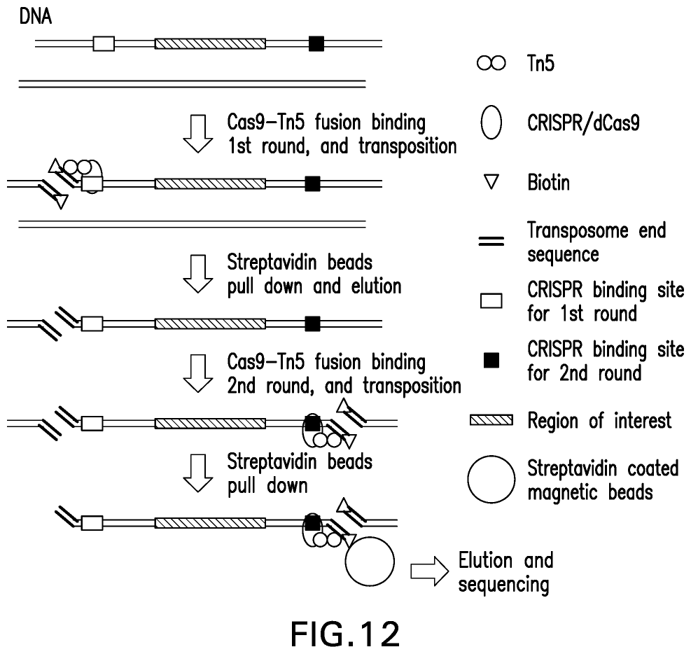

### DETAILED DESCRIPTION OF THE INVENTION

Before the present disclosure is further described, it is to be understood that this disclosure is not strictly limited to particular embodiments described herein, as such can of course vary. It is also to be understood that the terminology used herein is for the purpose of describing particular embodiments only, and is not intended to be limiting, since the scope of the present disclosure will be limited only by the claims.

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably.

As used in this application, the term “or” can be understood to mean “and/or.” In this application, the terms “comprising” and “including” can be understood to encompass itemized components or steps whether presented by themselves or together with one or more additional components or steps. Where ranges are provided herein, the endpoints are included. As used in this application, the term “comprise” and variations of the term, such as “comprising” and “comprises,” are not intended to exclude other additives, components, integers or steps.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the detailed methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided can be different from the actual publication dates, which can need to be independently confirmed.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments are specifically embraced by the present disclosure and are disclosed herein just as if each and every combination was individually and explicitly disclosed. In addition, all sub-combinations are also specifically embraced by the present disclosure and are disclosed herein just as if each and every such sub-combination was individually and explicitly disclosed herein.

It is further noted that the claims can be drafted to exclude any optional element. As such, this statement is intended to serve as antecedent basis for use of such exclusive terminology as “solely,” “only” and the like in connection with the recitation of claim elements or use of a “negative” limitation.

To further improve the enrichment factor of CRISPR/Cas system, described herein are new tandem approaches. If each specific Cas protein-DNA interaction and the following separation only yield a few hundred fold enrichment, when two such interactions and separations are combined together, the combined enrichment will be greater than ˜100*100=10,000 fold. In some embodiments, the two different Cas protein-DNA interactions are not using the same Cas protein or the same gRNA, in order to gain further selectivity or specificity. The detailed tandem approaches are described below.

Disclosed herein are methods of enriching for target nucleic acid molecules, comprising (a) binding target nucleic acid molecules in a sample with a first Cas protein/gRNA complex that is specific to a first locus of a target region of the target nucleic acid molecules; (b) separating the target nucleic acid molecules of (a) from nontarget nucleic acid molecules in the sample; and (c) binding the separated target nucleic acid molecules of (b) with a second Cas protein/gRNA complex that is specific to a second locus of the target region of the target nucleic acid molecules.

In some embodiments, the ends of the target and/or nontarget nucleic acid molecules in the sample are blocked by dephosphorylation, attaching a hairpin oligonucleotide, or nucleotide addition before the binding with the first and/or the second Cas protein/gRNA complex. In some embodiments, the ends of the target and nontarget nucleic acid molecules in the sample are blocked by dephosphorylation, attaching a hairpin oligonucleotide, or nucleotide addition before the binding with the first or the second Cas protein/gRNA complex.

The methods disclosed herein can further comprise separating the target nucleic acid molecules of (c) from nontarget nucleic acid molecules.

In some embodiments, the first locus is different from the second locus.

In some embodiments, the first Cas protein/gRNA complex comprises an active Cas protein and the second Cas protein/gRNA complex comprises an active Cas protein. In some embodiments, the first Cas protein/gRNA complex comprises an active Cas protein and the second Cas protein/gRNA complex comprises an inactive Cas protein. In other embodiments, the first Cas protein/gRNA complex comprises an inactive Cas protein and the second Cas protein/gRNA complex comprises an active Cas protein. In some embodiments, the first Cas protein/gRNA complex comprises an inactive Cas protein and the second Cas protein/gRNA complex comprises an inactive Cas protein.

The active Cas protein can cut the target nucleic acid molecules.

The methods disclosed herein can further comprise ligating an adapter oligonucleotide to the cut ends of the target nucleic acid molecules. In some embodiments, the adapter oligonucleotide, the Cas protein, the gRNA, or the target nucleic acid molecules are attached to an affinity label.

In some embodiments, the methods further comprise attaching an affinity label to the adapter oligonucleotide, the Cas protein, the gRNA, or the target nucleic acid molecules.

The affinity label can be an affinity tag for binding to a solid surface, His tag, TAP tag, or antibody.

In some embodiments, the separating is performed by binding the target nucleic acid molecules bound to the affinity label to an affinity label partner and eluting the bound target nucleic acid molecules.

The adapter oligonucleotide ligated to the target nucleic acid molecules cut by the first Cas protein/gRNA complex can be attached to an affinity label.

The methods can further comprise eluting the target nucleic acid molecules bound to the affinity label to an affinity label partner before the binding in (c).

In some embodiments, the inactive Cas protein or gRNA is attached to an affinity label.

The methods can further comprise binding the inactive Cas protein or gRNA attached to an affinity label to an affinity label partner and eluting the inactive Cas protein bound target nucleic acid molecules.

In some embodiments, the affinity label is an anti-dCas antibody linked to a bead.

The first Cas protein/gRNA complex can comprise 2, 3, 4, 5 or more active Cas protein/gRNA complexes or inactive Cas protein/gRNA complexes that are specific to a set of first loci of 2, 3, 4, 5 or more different target regions, respectively.

The second Cas protein/gRNA complex can comprise 2, 3, 4, 5 or more active Cas protein/gRNA complexes or inactive Cas protein/gRNA complexes that are specific to a set of second loci of 2, 3, 4, 5 or more different target regions, respectively.

The first Cas protein/gRNA complex can comprise 2, 3, 4, 5 or more inactive Cas protein/gRNA complexes that are specific to a set of first loci of 2, 3, 4, 5 or more different target regions, respectively.

The second Cas protein/gRNA complex can comprise 2, 3, 4, 5 or more inactive Cas protein/gRNA complexes that are specific to a set of second loci of 2, 3, 4, 5 or more different target regions, respectively.

Also disclosed herein are methods of enriching for target nucleic acid molecules, comprising (a) binding target nucleic acid molecules in a sample with a Cas protein/gRNA complex specific to a first locus of a target region of the target nucleic acid molecules; (b) binding target nucleic acid molecules in the sample with a Cas protein/gRNA complex specific to a second locus of a target region of the target nucleic acid molecules; and (c) separating the target nucleic acid molecules from nontarget nucleic acid molecules in the sample.

In some embodiments, the ends of the target and/or nontarget nucleic acid molecules in the sample are blocked by dephosphorylation, attaching a hairpin oligonucleotide, or nucleotide addition before the binding with the Cas protein/gRNA complex in (a) and/or (b). In some embodiments, the ends of the target and nontarget nucleic acid molecules in the sample are blocked by dephosphorylation, attaching a hairpin oligonucleotide, or nucleotide addition before the binding with the Cas protein/gRNA complex in (a) or (b).

In some embodiments, the first locus of the target region is bound by a Cas protein/gRNA complex comprising an active Cas protein and the second locus of the target region is bound by a Cas protein/gRNA complex comprising an inactive Cas protein. The first locus of the target region can be bound by a Cas protein/gRNA complex comprising an inactive Cas protein and the second locus of the target region can be bound by a Cas protein/gRNA complex comprising an active Cas protein. The active and inactive Cas protein/gRNA complexes can bind to the target nucleic acid molecules in a same reaction.

The active Cas protein/gRNA complex can cut the target nucleic acid molecules.

The methods can further comprise ligating an adapter oligonucleotide to cut ends of the target nucleic acid molecules. In some embodiments, the adapter oligonucleotide, the Cas protein, the gRNA, or the target nucleic acid molecules are attached to an affinity label. In some embodiments, the methods further comprise attaching an affinity label to the adapter oligonucleotide, the Cas protein, the gRNA, or the target nucleic acid molecules. The affinity label can be an affinity tag for binding to a solid surface, His tag, TAP tag, or antibody.

In some embodiments, the separating is performed by binding the target nucleic acid molecules bound to the affinity label to an affinity label partner and eluting the bound target nucleic acid molecules.

In some embodiments, the first Cas protein/gRNA complex comprises 2, 3, 4, 5 or more active Cas protein/gRNA complexes or inactive Cas protein/gRNA complexes that are specific to a set of first loci of 2, 3, 4, 5 or more different target regions, respectively. In some embodiments, the second Cas protein/gRNA complex comprises 2, 3, 4, 5 or more active Cas protein/gRNA complexes or inactive Cas protein/gRNA complexes that are specific to a set of second loci of 2, 3, 4, 5 or more different target regions, respectively.

Further disclosed herein are methods of enriching for target nucleic acid molecules, comprising: (a) binding target nucleic acid molecules in a sample with one or more first target endonucleases that are specific to a first locus of a target region of the target nucleic acid molecules; (b) separating the target nucleic acid molecules from nontarget nucleic acid molecules in the sample; and (c) binding the separated target nucleic acid molecules with one or more second target endonucleases that are specific to a second locus of the target region of the target nucleic acid molecules.

In some embodiments, the ends of the target and/or nontarget nucleic acid molecules in the sample can be blocked by dephosphorylation, attaching a hairpin oligonucleotide, or nucleotide addition before the binding with the first and/or the second target endonucleases. In some embodiments, the ends of the target and nontarget nucleic acid molecules in the sample can be blocked by dephosphorylation, attaching a hairpin oligonucleotide, or nucleotide addition before the binding with the first or the second target endonucleases.

The methods can further comprise separating the target nucleic acid molecules of (c) from nontarget nucleic acid molecules.

The methods can further comprise binding the separated target nucleic acid molecules with one or more third target endonucleases that are specific to a third locus of the target region and separating the target nucleic acid molecules from nontarget nucleic acid molecules.

The first target endonucleases and the second target endonucleases can target different loci of the target region. The first target endonucleases, the second target endonucleases, and the third target endonucleases can target different loci of the target region. The methods can enrich for multiple target regions.

The methods can further comprise releasing the target nucleic acid molecules from the first or second target endonucleases.

The one or more target endonucleases can be an active or inactive Cas protein, a Cas9-like enzyme, a Cpfl enzyme, a ribonucleoprotein, a meganuclease, a transcription activator-like effector-based nuclease (TALEN), a zinc-finger nuclease, an argonaute nuclease, a megaTAL nuclease, or a combination thereof. The one or more target endonucleases can be Cas9, CPFl, or a derivative thereof. The target endonucleases can be active or inactive Cas enzyme or Cpfl enzyme.

The binding can comprise cutting the nucleic acid molecules with the target endonucleases.

The target endonucleases can comprise 2, 3, 4, 5 or more Cas enzymes that bind to 2, 3, 4, 5 or more different loci in the same target region or different target regions, respectively. The Cas enzymes can comprise the same type of Cas enzyme. The Cas enzymes can comprise two or more different types of Cas enzymes.

In some embodiments, the separating the target nucleic acid molecules from the nontarget nucleic acid molecules can comprise gel electrophoresis, gel purification, liquid chromatography, size exclusion purification, filtration, SPRI bead purification, or enzymatic digestion of the nontarget nucleic acid molecules.

The methods can further comprise ligating an adapter to at least one of the 5′ or 3′ ends of the cut target nucleic acid molecules.

A transposase can be tethered to the first or second target endonuclease and the tethered transposase inserts a transposon end sequence tag in or near the binding site of the endonuclease. The transposase can be tethered to the target endonuclease through protein fusion.

The one or more target endonucleases can remain bound to the target region of the target nucleic acid molecules.

At least one target endonuclease or adapter can be attached to an affinity label. The affinity label is or comprises at least one of Acrydite, azide, azide (NHS ester), digoxigenin (NHS ester), Thinker, Amino modifier C6, Amino modifier C12, Amino modifier C6 dT, Unilink amino modifier, hexynyl, 5-octadiynyl dU, biotin, biotin (azide), biotin dT, biotin TEG, dual biotin, PC biotin, desthiobiotin TEG, thiol modifier C3, dithiol, thiol modifier C6 S-S, and/or succinyl groups.

The affinity label can be an affinity tag for binding to a solid surface, His tag, TAP tag, or antibody.

The methods can further comprise capturing the target nucleic acid molecules with an affinity label partner. The affinity label partner is or comprises at least one of amino silane, epoxy silane, isothiocyanate, aminophenyl silane, aminpropyl silane, mercapto silane, aldehyde, epoxide, phosphonate, streptavidin, avidin, an antibody, a hapten recognizing an antibody, a particular nucleic acid sequence, magnetically attractable particles (e.g., Dynabeads), and/or photolabile resins.

The methods can further comprise analyzing the target nucleic acid molecules. The analyzing can comprise quantitation and/or sequencing of the target region. The quantitation can comprise at least one of spectrophotometric analysis, real-time PCR, and/or fluorescence-based quantitation. The sequencing can comprise next-generation sequencing, third-generation sequencing, duplex sequencing, SPLiT-duplex sequencing, Sanger sequencing, shotgun sequencing, bridge amplification/sequencing, nanopore sequencing, single molecule real-time sequencing, ion torrent sequencing, pyrosequencing, digital sequencing, direct digital sequencing, sequencing by ligation, polony-based sequencing, electrical current-based sequencing, sequencing via mass spectroscopy, microfluidics-based sequencing, and combinations thereof.

### Definitions

The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” can encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

The term “sample” can include nucleic acid molecules, such as RNA or DNA, a single cell, multiple cells, fragments of cells, or an aliquot of body fluid. In some embodiments, the sample can be taken from a subject or patient (e.g., a mammalian subject, an animal subject, a human subject, or a nonhuman animal subject). Samples can be selected by one of skill in the art using any known means known including but not limited to centrifugation, venipuncture, blood draw, excretion, swabbing, biopsy, needle aspirate, lavage sample, scraping, surgical incision, laser capture microdissection, gradient separation, or intervention or other means known in the art. The term “mammal” or “mammalian” as used herein includes both humans and nonhumans and include but is not limited to humans, nonhuman primates, canines, felines, murines, bovines, equines, and porcines.

As used herein, the term “biological sample” is intended to include, but is not limited to, tissues, cells, biological fluids and isolates thereof, isolated from a subject, as well as tissues, cells, and fluids present within a subject.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

A single cell suspension can be obtained using standard methods known in the art including, for example, enzymatically using trypsin or papain to digest proteins connecting cells in tissue samples or releasing adherent cells in culture, or mechanically separating cells in a sample. Samples can also be selected by one of skill in the art using one or more markers known to be associated with a sample of interest.

Methods for manipulating single cells are known in the art and include fluorescence activated cell sorting (FACS), micromanipulation and the use of semi-automated cell pickers (e.g., the Quixell™ cell transfer system from Stoelting Co.). Individual cells can, for example, be individually selected based on features detectable by microscopic observation, such as location, morphology, or reporter gene expression.

Once a desired sample has been identified, the sample is prepared and the cell(s) are lysed to release cellular contents including DNA and RNA, such as gDNA and mRNA, using methods known to those of skill in the art. Lysis can be achieved by, for example, heating the cells, or by the use of detergents or other chemical methods, or by a combination of these. Any suitable lysis method known in the art can be used. Methods for preparation of samples comprising nucleic acid molecules are well known in the art. See also WO2019/191122.

“Multiple samples” means more than one sample, such as but not limited to 2 or more, 3 or more, 4 or more, 5 or more, 2-5, 6-10, 11-15, 16-20, 21-30, 31-40, 41-50, 51-100, more than 100, or any specific number or ranges of samples derived therefrom. The multiple samples can be derived from one source or origin or from different sources or origins.

The term “polynucleotide(s)” or “oligonucleotide(s)” refers to nucleic acids such as DNA molecules and RNA molecules and analogs thereof (e.g., DNA or RNA generated using nucleotide analogs or using nucleic acid chemistry). As desired, the polynucleotides can be made synthetically, e.g., using art-recognized nucleic acid chemistry or enzymatically using, e.g., a polymerase, and, if desired, can be modified. Typical modifications include methylation, biotinylation, and other art-known modifications. In addition, a polynucleotide can be single-stranded or double-stranded and, where desired, linked to a detectable moiety. In some aspects, a polynucleotide can include hybrid molecules, e.g., comprising DNA and RNA.

“G,” “C,” “A,” “T” and “U” each generally stands for a nucleotide that contains guanine, cytosine, adenine, thymidine and uracil as a base, respectively. However, it will be understood that the term “ribonucleotide” or “nucleotide” can also refer to a modified nucleotide or a surrogate replacement moiety. The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in nucleotide sequences by a nucleotide containing, for example, inosine. In another example, adenine and cytosine anywhere in the oligonucleotide can be replaced with guanine and uracil, respectively, to form G-U Wobble base pairing with the target mRNA. Sequences containing such replacement moieties are suitable for the compositions and methods described herein.

As used herein, the term “nucleotide analogs” refers to synthetic analogs having modified nucleotide base portions, modified pentose portions, and/or modified phosphate portions, and, in the case of polynucleotides, modified internucleotide linkages, as generally described elsewhere (e.g., Scheit, Nucleotide Analogs, John Wiley, New York, 1980; Englisch, Angew. Chem. Int. Ed. Engl. 30:613-29, 1991; Agarwal, Protocols for Polynucleotides and Analogs, Humana Press, 1994; and S. Verma and F. Eckstein, Ann. Rev. Biochem. 67:99-134, 1998). Exemplary phosphate analogs include but are not limited to phosphorothioate, phosphorodithioate, phosphoroselenoate, phosphorodiselenoate, phosphoroanilothioate, phosphoranilidate, phosphoramidate, boronophosphates, including associated counterions, e.g., H+, NH4+, Na+, if such counterions are present. Exemplary modified nucleotide base portions include but are not limited to 5-methylcytosine (5mC); C-5-propynyl analogs, including but not limited to, C-5 propynyl-C and C-5 propynyl-U; 2,6-diaminopurine, also known as 2-amino adenine or 2-amino-dA); hypoxanthine, pseudouridine, 2-thiopyrimidine, isocytosine (isoC), 5-methyl isoC, and isoguanine (isoG; see, e.g., U.S. Pat. No. 5,432,272). Exemplary modified pentose portions include but are not limited to, locked nucleic acid (LNA) analogs including without limitation Bz-A-LNA, 5-Me-Bz-C-LNA, dmf-G-LNA, and T-LNA (see, e.g., The Glen Report, 16(2):5, 2003; Koshkin et al., Tetrahedron 54:3607-30, 1998), and 2′- or 3′-modifications where the 2′- or 3′-position is hydrogen, hydroxy, alkoxy (e.g., methoxy, ethoxy, allyloxy, isopropoxy, butoxy, isobutoxy and phenoxy), azido, amino, alkylamino, fluoro, chloro, or bromo. Modified internucleotide linkages include phosphate analogs, analogs having achiral and uncharged intersubunit linkages (e.g., Sterchak, E. P. et al., Organic Chern., 52:4202, 1987), and uncharged morpholino-based polymers having achiral intersubunit linkages (see, e.g., U.S. Pat. No. 5,034,506). Some internucleotide linkage analogs include morpholidate, acetal, and polyamide-linked heterocycles.

The term “DNA” refers to chromosomal DNA, plasmid DNA, phage DNA, or viral DNA that is single stranded or double stranded. DNA can be obtained from prokaryotes or eukaryotes.

The term “genomic DNA” or gDNA” refers to chromosomal DNA.

The term “messenger RNA” or “mRNA” refers to an RNA that is without introns and that can be translated into a polypeptide.

The term “cDNA” refers to a DNA that is complementary or identical to an mRNA, in either single stranded or double stranded form.

The term “target nucleic acid molecule(s)” or “target nucleic acid” is intended to mean a nucleic acid molecule(s) that is the object of an analysis or action. The analysis or action includes subjecting the nucleic acid molecule(s) to copying, amplification, sequencing and/or other procedure for nucleic acid interrogation. A target nucleic acid can include nucleotide sequences additional to the target sequence to be analyzed. For example, a target nucleic acid can include one or more adapters, including an adapter that functions as a primer binding site, that flank(s) a target nucleic acid sequence that is to be analyzed. A target nucleic acid hybridized to a capture oligonucleotide or capture primer can contain nucleotides that extend beyond the 5′ or 3′ end of the capture oligonucleotide in such a way that not all of the target nucleic acid is amenable to extension.

The term “nontarget nucleic acid molecule(s)” or “nontarget nucleic acid” means nucleic acid molecule(s) that is the not the object of an analysis or action, e.g., from which the target nucleic acid molecule(s) are separated, physically or virtually.

The term “target specific” or “specific to” when used in reference to a guide RNA, a crRNA or a derivative thereof, or other nucleotide is intended to mean a polynucleotide that includes a nucleotide sequence specific to a target polynucleotide sequence, namely a sequence of nucleotides capable of selectively annealing to an identifying region of a target polynucleotide, i.e., a target region of a target nucleic acid molecule. Target specific nucleotides can have a single species of oligonucleotides, or it can include two or more species with different sequences. Thus, the target specific nucleotides can be two or more sequences, including 3, 4, 5, 6, 7, 8, 9 or 10 or more different sequences. In some embodiments, a crRNA or the derivative thereof contains a target-specific nucleotide region complementary to a target sequence in a target region of the target nucleic acid molecule. In some embodiments, a crRNA or the derivative thereof can contain other nucleotide sequences besides a target-specific nucleotide region. In some embodiments, the other nucleotide sequences can be from a tracrRNA sequence.

As used herein, the term “complementary” when used in reference to a polynucleotide is intended to mean a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968). Annealing conditions will depend upon the particular application and can be routinely determined by persons skilled in the art, without undue experimentation.

As used herein, the term “hybridization” refers to the process in which two single-stranded polynucleotides bind noncovalently to form a stable double-stranded polynucleotide. A resulting double-stranded polynucleotide is a “hybrid” or “duplex.” Hybridization conditions will typically include salt concentrations of less than about 1 M, more usually less than about 500 mM and can be less than about 200 mM. A hybridization buffer includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are usually performed under stringent conditions, i.e., conditions under which a probe will hybridize to its target subsequence but will not hybridize to other, noncomplementary sequences (in nontarget nucleic acid molecules). Stringent conditions are sequence-dependent and are different in different circumstances, and can be determined routinely by those skilled in the art.

As used herein, the terms “ligating,” “ligation,” and their derivatives refer generally to the act or process for covalently linking two or more molecules together, for example, covalently linking two or more nucleic acid molecules to each other. In some embodiments, ligation includes joining nicks between adjacent nucleotides of nucleic acids. In some embodiments, ligation includes forming a covalent bond between an end of a first and an end of a second nucleic acid molecule. In some embodiments, for example embodiments wherein the nucleic acid molecules to be ligated include conventional nucleotide residues, the litigation can include forming a covalent bond between a 5′ phosphate group of one nucleic acid and a 3′ hydroxyl group of a second nucleic acid thereby forming a ligated nucleic acid molecule. In some embodiments, any means for joining nicks or bonding a 5′phosphate to a 3′ hydroxyl between adjacent nucleotides can be employed. In an exemplary embodiment, an enzyme such as a ligase can be used. Generally, for the purposes of this disclosure, an amplified target sequence can be ligated to an adapter to generate an adapter-ligated amplified target sequence.

As used herein, “ligase” and its derivatives, refers generally to any agent capable of catalyzing the ligation of two substrate molecules. In some embodiments, the ligase includes an enzyme capable of catalyzing the joining of nicks between adjacent nucleotides of a nucleic acid. In some embodiments, the ligase includes an enzyme capable of catalyzing the formation of a covalent bond between a 5′ phosphate of one nucleic acid molecule to a 3′ hydroxyl of another nucleic acid molecule thereby forming a ligated nucleic acid molecule. Suitable ligases can include, but not limited to, T4 DNA ligase, T4 RNA ligase, and E. coli DNA ligase.

As used herein, “ligation conditions” and its derivatives, generally refers to conditions suitable for ligating two molecules to each other. In some embodiments, the ligation conditions are suitable for sealing nicks or gaps between nucleic acids. As defined herein, a “nick” or “gap” refers to a nucleic acid molecule that lacks a directly bound 5′ phosphate of a mononucleotide pentose ring to a 3′ hydroxyl of a neighboring mononucleotide pentose ring within internal nucleotides of a nucleic acid sequence. As used herein, the term nick or gap is consistent with the use of the term in the art. Typically, a nick or gap can be ligated in the presence of an enzyme, such as ligase at an appropriate temperature and pH. In some embodiments, T4 DNA ligase can join a nick between nucleic acids at a temperature of about 70° C.-72° C.

As used herein, “blunt-end ligation” and its derivatives, refers generally to ligation of two blunt-end double-stranded nucleic acid molecules to each other. A “blunt end” refers to an end of a double-stranded nucleic acid molecule wherein substantially all of the nucleotides in the end of one strand of the nucleic acid molecule are base paired with opposing nucleotides in the other strand of the same nucleic acid molecule. A nucleic acid molecule is not blunt ended if it has an end that includes a single-stranded portion greater than two nucleotides in length, referred to herein as an “overhang.” In some embodiments, the end of nucleic acid molecule does not include any single stranded portion, such that every nucleotide in one strand of the end is based paired with opposing nucleotides in the other strand of the same nucleic acid molecule. In some embodiments, the ends of the two blunt ended nucleic acid molecules that become ligated to each other do not include any overlapping, shared or complementary sequence. Typically, blunted-end ligation excludes the use of additional oligonucleotide adapters to assist in the ligation of the double-stranded amplified target sequence to the double-stranded adapter, such as patch oligonucleotides as described in Mitra and Varley, US2010/0129874. In some embodiments, blunt-ended ligation includes a nick translation reaction to seal a nick created during the ligation process.

As used herein, in its broadest sense, “nucleic acid molecule(s)” or “nucleic acid” refers to any compound and/or substance is or can be incorporated into an oligonucleotide chain. In some embodiments, nucleic acid molecules are obtained from samples and comprise target nucleic acid molecules and nontarget nucleic acid molecules. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids,” which are known in the art and have peptide bonds instead of phosphodiester bonds in the backbone, are considered within the scope of the present technology. Alternatively, or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxyguanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, CS-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, hexose or Locked Nucleic acids) as compared with those in commonly occurring natural nucleic acids. In some embodiments, a nucleic acid has a nucleotide sequence that encodes a functional gene product such as an RNA or protein. In some embodiments, a nucleic acid includes one or more introns. In some embodiments, a nucleic acid can be a nonprotein coding RNA product, such as a microRNA, a ribosomal RNA, or a CRISPR/Cas guide RNA. In some embodiments, a nucleic acid serves a regulatory pUipose in a genome. In some embodiments, a nucleic acid does not arise from a genome. In some embodiments, a nucleic acid includes intergenic sequences. In some embodiments, a nucleic acid derives from an extrachromosomal element or a non-nuclear genome (mitochondrial, chloroplast etc.), In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double-stranded. In some embodiments a nucleic acid has a nucleotide sequence comprising at least one element that encodes, or is the complement of a sequence that encodes, a polypeptide. In some embodiments, a nucleic acid has enzymatic activity. In some embodiments the nucleic acid serves a mechanical function, for example in a ribonucleoprotein complex or a transfer RNA. In some embodiments a nucleic acid function as an aptamer. In some embodiments a nucleic acid can be used for data storage. In some embodiments, a nucleic acid can be chemically synthesized in vitro.

As used herein, the term “subject” refers an organism, typically a mammal (e.g., a nonhuman or human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, in the context of enriching a target nucleic acid molecule, the term “enrich,” “enriching”, or “enrichment” refers to a process which results in a higher percentage of the target nucleic acid molecules in a polynucleotide population or samples containing target nucleic acid molecules and nontarget nucleic acid molecules. In some embodiments, the percentage increases about 5% or more, 10% or more, 20% or more, 30% or more, 40% or more, 50% or more, 60% or more, 70% or more, 80% or more, 90% or more, 100%, or any ranges derived therefrom. In some embodiments, the percentage increases about 2 fold or more, 5 fold or more, 10 fold or more, 50 fold or more, 100 fold or more, 150 fold or more, 200 fold or more, 500 fold or more, 1000 fold or more, 5000 fold or more, 10000 fold or more, or any ranges derived therefrom, e.g., compared to prior CRISPR/Cas enrichment methods that are not tandem as disclosed herein.

### Targeted Endonucleases

Various aspects of the disclosure herein include enrichment of target nucleic acid molecules using adapters, oligonucleotides, and capture labels that can incorporate enzymatic cleavage, enzymatic cleavage of a single strand, enzymatic cleavage of double strands, incorporation of a modified nucleic acid followed by enzymatic treatment that leads to cleavage or one or both strands, incorporation of a photocleavable linker, incorporation of a uracil, incorporation of a ribose base, incorporation of an 8-oxo-guanine adduct, use of a restriction endonuclease, use of site-directed cutting enzymes, and the like. In other embodiments, endonucleases, such as a ribonucleoprotein endonuclease (e.g., an active or inactive Cas protein, such as active or inactive Cas9 or CPFl), or other programmable endonuclease (e.g., a homing endonuclease, a zinc-fingered nuclease, a TALEN, a meganuclease (e.g., megaTAL nuclease), an argonaute nuclease, etc.), and any combinations thereof can be used.

The term “locus” or “loci” refers to a position on a nucleic acid molecule where a specific sequence or other genetic marker is located.

The term “target region” or “target sequence” refers to a sequence in a double-stranded DNA molecule, where the target sequence is bound, and, optionally cleaved or nicked by a targeted endonuclease, e.g., Cas protein or bound by inactive Cas protein (dCas). Thus, the target region or sequence contains the binding site or locus to which the targeted endonuclease, e.g., Cas protein/gRNA complex, binds. The target region or sequence is contained in the target nucleic acid or nucleic acid molecule. In many cases, a target sequence can be unique in any one starting molecule and, as will be described in greater detail below, multiple different starting molecules (e.g., overlapping fragments) can contain the same target sequence. In some cases, the target sequence can be degenerate, that is, the target sequence can have base positions that can have variable bases. These positions can be denoted as Y, R, N, etc., where Y and R denote pyrimidine and purine bases, respectively, and N denotes any of the 4 bases.

The term “cleaving,” as used herein, refers to a reaction that breaks the phosphodiester bonds between two adjacent nucleotides in both strands of a double-stranded DNA molecule, thereby resulting in a double-stranded break in the DNA molecule.

The term “nicking,” as used herein, refers to a reaction that breaks the phosphodiester bond between two nucleotides in one strand of a double-stranded DNA molecule to produce a 3′ hydroxyl group and a 5′ phosphate group.

The terms “cleavage site,” and “nick site,” as used herein, refers to the site at which a double-stranded DNA molecule has been cleaved or nicked.

Targeted endonucleases (e.g., a CRISPR-associated ribonucleoprotein complex, such as Cas9 enzyme, a Cas9-like enzyme, a Cpfl enzyme, a ribonucleoprotein, a meganuclease, a transcription activator-like effector-based nuclease (TALEN), a zinc-finger nuclease, an argonaute nuclease, a megaTAL nuclease, or a combination thereof) can be used to selectively cut and excise targeted portions of target nucleic acid molecules for purposes of enriching such targeted portions for sequencing applications. In some embodiments, a targeted endonuclease can be modified, such as having an amino acid substitution for provided, for example, enhanced thermostability, salt tolerance and/or pH tolerance or enhanced specificity or alternate PAM site recognition or higher affinity for binding. In other embodiments, a targeted endonuclease can be biotinylated, fused with streptavidin and/or incorporate other affinity-based (e.g., bait/prey) technology. In certain embodiments, a targeted endonuclease can have an altered recognition site specificity (e.g., SpCas9 variant having altered PAM site specificity). In other embodiments, a targeted endonuclease can be catalytically inactive so that cleavage does not occur once bound to targeted portions of nucleic acid molecules. In some embodiments, a targeted endonuclease is modified to cleave a single strand of a targeted portion of nucleic acid molecules (e.g., a nickase variant) thereby generating a nick in the nucleic acid molecules. CRISPR-based targeted endonucleases are further discussed herein to provide a further detailed nonlimiting example of use of a targeted endonuclease. The nomenclature around such targeted nucleases remains in flux. For purposes herein, the term “CRISPR-based” generally means Cas proteins or endonucleases comprising a nucleic acid sequence, the sequence of which can be modified to redefine a nucleic acid sequence to be cleaved. Cas9 and CPFl are examples of such targeted endonucleases currently in use, but many more appear to exist in different places in the natural world and the availability of different varieties of such targeted and easily tunable nucleases is expected to grow rapidly in the coming years. For example, Cas12a, Cas13, CasX and others are contemplated for use in various embodiments. Similarly, multiple engineered variants of these enzymes to enhance or modify their properties are becoming available. Explicitly contemplate herein are uses of substantially functionally similar targeted endonucleases not explicitly described herein or not yet discovered, to achieve a similar purpose to disclosures described within.

### Ligateable Ends

In some embodiments, adapters or adapter oligonucleotides are generated with a ligateable 3′ end suitable for ligation to target double-stranded nucleic acid sequences (e.g., for sequencing library preparation). Ligation domains present in each of the double-stranded adapter products can be capable of being ligated to one corresponding strand of a double-stranded target or nontarget nucleic acid sequence. In some embodiments, one of the ligation domains includes a T-overhang, an A-overhang, a CG-overhang, a multiple nucleotide overhang, a blunt end, or another ligateable nucleic acid sequence. In some embodiments, a double-stranded 3′ ligation domain comprises a blunt end. In certain embodiments, at least one of the ligation domain sequences includes a modified or nonstandard nucleic acid. In some embodiments, a modified nucleotide can be an abasic site, a uracil, tetrahydrofuran, 8-oxo-7,8-dihydro-2′-deoxyadenosine (8-oxo-A), 8-oxo-7,8-dihydro-2′-deoxyguanosine (8-oxo-G), deoxyinosine, 5′-nitroindole, 5-Hydroxymethyl-2′-deoxycytidine, iso-cytosine, 5′-methyl-isocytosine, or iso-guanosine. In some embodiments, at least one strand of the ligation domain includes a dephosphorylated base. In some embodiments, at least one of the ligation domains includes a dehydroxylated base. In some embodiments, at least one strand of the ligation domain has been chemically modified so as to render it unligateable (e.g., until a further action is performed to render the ligation domain ligateable). In some embodiments a 3′ overhang is obtained by use of a polymerase with terminal transferase activity. In some embodiments, Taq polymerase can add a single base pair overhang.

### Adapters and Adapter Sequences

In various arrangements, adapter or adapter nucleotide molecules that comprise molecular barcodes, primer sites, flow cell sequences and/or other features are contemplated for use with many of the embodiments disclosed herein. In some embodiments, provided adapters can be or comprise one or more sequences complementary or at least partially complementary to PCR primers (e.g., primer sites) that have at least one of the following properties: 1) high target specificity; 2) capable of being multiplexed; and 3) exhibit robust and minimally biased amplification.

In some embodiments, adapter molecules can be “linear,” “Y”-shaped, “U”-shaped, “hairpin” shaped, have a bubble (e.g., a portion of sequence that is noncomplementary), or other features. In other embodiments, adapter molecules can comprise a “Y”-shape, a “U”-shaped, a “hairpin” shaped, or a bubble. Certain adapters can comprise modified or nonstandard nucleotides, restriction sites, or other features for manipulation of structure or function in vitro. Adapter molecules can ligate to a variety of nucleic acid molecules having a terminal end. For example, adapter molecules can be suited to ligate to a T-overhang, an A-overhang, a CG-overhang, a multiple nucleotide overhang (also referred to herein as a “sticky end” or “sticky overhang”), a dehydroxylated base, a blunt end of a nucleic acid molecules and the end of a molecule were the 5′ of the target is dephosphorylated or otherwise blocked from traditional ligation. In other embodiments the adapter molecule can contain a dephosphorylated or otherwise ligation-preventing modification on the 5′ strand at the ligation site. In the latter two embodiments such strategies can be useful for preventing dimerization of library fragments or adapter molecules.

In some embodiments, adapter molecules can comprise a capture moiety suitable for isolating a desired target nucleic acid molecule ligated thereto.

An adapter sequence can mean a single-strand sequence, a double-strand sequence, a complementary sequence, a noncomplementary sequence, a partial complementary sequence, an asymmetric sequence, a primer binding sequence, a flow-cell sequence, a ligation sequence or other sequence provided by an adapter molecule. In particular embodiments, an adapter sequence can mean a sequence used for amplification by way of complement to an oligonucleotide.

In some embodiments, the disclosed methods and compositions include at least one adapter sequence (e.g., two adapter sequences, one on each of the 5′ and 3′ ends of a nucleic acid molecules). In some embodiments, the disclosed methods and compositions can comprise 2 or more adapter sequences (e.g., 3, 4, 5, 6, 7, 8, 9, 10 or more). In some embodiments, at least two of the adapter sequences differ from one another (e.g., by sequence). In some embodiments, each adapter sequence differs from each other adapter sequence (e.g., by sequence). In some embodiments, at least one adapter sequence is at least partially noncomplementary to at least a portion of at least one other adapter sequence (e.g., is noncomplementary by at least one nucleotide).

In some embodiments, an adapter sequence comprises at least one nonstandard nucleotide. In some embodiments, a nonstandard nucleotide is selected from an abasic site, a uracil, tetrahydrofuran, 8-oxo-7,8-dihydro-2′deoxyadenosine (8-oxo-A), 8-oxo-7,8-dihydro-2′-deoxyguanosine (8-oxo-G), deoxyinosine, 5′nitroindole, 5-Hydroxymethyl-2′-deoxycytidine, iso-cytosine, 5′-methyl-isocytosine, or isoguanosine, a methylated nucleotide, an RNA nucleotide, a ribose nucleotide, an 8-oxo-guanine, a photocleavable linker, a biotinylated nucleotide, a desthiobiotin nucleotide, a thiol modified nucleotide, an acrydite modified nucleotide an iso-dC, an iso-dG, a 2′-O-methyl nucleotide, an inosine nucleotide Locked Nucleic Acid, a peptide nucleic acid, a 5-methyl dC, a 5-bromodeoxyuridine, a 2,6-Diaminopurine, 2-Aminopurine nucleotide, an abasic nucleotide, a 5-Nitroindole nucleotide, an adenylated nucleotide, an azide nucleotide, a digoxigenin nucleotide, an I-linker, an 5′-Hexynyl modified nucleotide, a 5-Octadiynyl dU, photocleavable spacer, a non-photocleavable spacer, a click chemistry compatible modified nucleotide, and any combination thereof.

In some embodiments, an adapter sequence comprises a moiety having a magnetic property (i.e., a magnetic moiety). In some embodiments this magnetic property is paramagnetic. In some embodiments where an adapter sequence comprises a magnetic moiety (e.g., a nucleic acid molecules ligated to an adapter sequence comprising a magnetic moiety), when a magnetic field is applied, an adapter sequence comprising a magnetic moiety is substantially separated from adapter sequences that do not comprise a magnetic moiety (e.g., a nucleic acid molecules ligated to an adapter sequence that does not comprise a magnetic moiety).

### Enrichment Using CRISPR/Cas Endonuclease System

In some aspects, disclosed herein are methods for enriching region(s) of interest, i.e., target nucleic acid molecules, using the Clustered Regularly Interspaced Short Palindromic Repeats (CRISPR) programmable endonuclease system. In other aspects, CRISPR-like or other programmable endonucleases such as zinc-finger nucleases, TALEN nucleases or other sequence-specific endonucleases such as homing endonucleases or simple restriction nucleases or derivatives thereof can be used alone or in combination as part of the disclosed technology.

In particular, CRISPR/Cas (or other programmable or nonprogrammable endonucleases or a combination thereof) can be used to selectively cleave a nucleic backbone in one or more defined or semi-defined region to functionally excise one or more sequence regions of interest from within a longer nucleic acid molecule, thus enabling enrichment of one or more nucleic acid target region of interest. In other embodiments, CRISPR/Cas (or other programmable endonuclease or nonprogrammable endonuclease or a combination thereof) can be used to selectively bind one or more sequence regions of interest. These programmable endonucleases can be used either alone or in combination with other forms of targeted nucleases, such as restriction endonuclease, or other enzymatic or nonenzymatic methods for cleaving nucleic acids.

As used herein, the term “CRISPR-Cas system” refers to an enzyme system including a guide RNA sequence that contains a nucleotide sequence complementary or substantially complementary to a region of a target polynucleotide, and a protein with nuclease activity. CRISPR-Cas systems include Type I CRISPR-Cas system, Type II CRISPR-Cas system, Type III CRISPR-Cas system, and derivatives thereof. CRISPR-Cas systems include engineered and/or programmed nuclease systems derived from naturally accruing CRISPR-Cas systems. CRISPR-Cas systems can contain engineered and/or mutated Cas proteins. CRISPR-Cas systems can contain engineered and/or programmed guide RNA.

The terms “Cas protein/gRNA” and “Cas-gRNA complex” refer to a complex comprising a Cas protein and a guide RNA (gRNA).

As used herein, “gRNA,” “guide RNA,” or “Cas9-associated guide RNA” refers to short RNA molecules which include a scaffold sequence suitable for a targeted endonuclease (e.g., an active or inactive Cas enzyme such as Cas9 or Cpfl or another ribonucleoprotein with similar properties, etc.) binding to a substantially target-specific sequence, which can then facilitate cutting of a specific region of DNA or RNA. gRNA can comprise a crRNA molecule and a tracrRNA molecule or a single molecule (i.e., a sgRNA) that contains both crRNA and tracrRNA sequences. The Cas9-associated guide RNA can exist as isolated RNA, or as part of a Cas9-gRNA complex.

Reference to a Cas9-associated guide RNA that is “complementary to” another sequence is not intended to mean that the entire guide RNA is complementary to the other sequence. A Cas9-associated guide RNA that is complementary to another sequence comprises a sequence that is complementary to the other sequence. Specifically, it is known that a Cas9 complex can specifically bind to a target sequence that has as few as 8 or 9 bases of complementarity with the guide Cas9-associated guide RNA in the complex. Off-site binding can be decreased by increasing the length of complementarity, e.g., to 15 or 20 bases.

The term “Cas protein,” “Cas enzyme,” or “Cas nuclease” refers to active or inactive Cas protein such as Cas9 or Cpfl or another ribonucleoprotein with similar properties that can bind to a substantially target-specific sequence that is determined by the guide RNA.

A Cas protein that is active has nuclease activity, e.g., has active HNH and RuvC nucleases. Such a protein can bind to a target site in double-stranded DNA (where the target site is determined by the guide RNA) and cleave or nick the double-stranded DNA.

A Cas protein that is deactivated or inactivated (also “dCas” or “inactivated Cas”) is a mutant Cas protein that has inactivated nuclease activity, e.g., has inactivated HNH and RuvC nucleases. Such a protein can bind to a target site in double-stranded DNA (where the target site is determined by the guide RNA), but the protein is unable to cleave or nick the double-stranded DNA.

In some embodiments, the Cas protein or the variant thereof is a Cas9 protein or a variant thereof. In some embodiments, the Cas9 protein is derived from Cas9 protein of S. thermophilus CRISPR-Cas system. In some embodiments, the Cas9 protein is a multi-domain protein of about 1,409 amino acids residues.

A Cas9 protein can be at least 60% identical (e.g., at least 70%, at least 80%, or 90% identical, at least 95% identical or at least 98% identical or at least 99% identical) to a wild type Cas9 protein, e.g., to the Streptococcus pyogenes Cas9 protein. The Cas9 protein can have all the functions of a wild type Cas 9 protein (active Cas9), or only one or some of the functions (inactive Cas9 or dCas9), including binding activity, nuclease activity, and nuclease activity.

For Cas9 to successfully bind to DNA, the target sequence in the genomic DNA (or target DNA or target nucleic acid molecule) should be complementary to the gRNA sequence and must be immediately followed by the correct protospacer adjacent motif or “PAM” sequence. The PAM sequence is present in the DNA target sequence but not in the gRNA sequence. Any DNA sequence with the correct target sequence followed by the PAM sequence will be bound by Cas9. The PAM sequence varies by the species of the bacteria from which Cas9 was derived. The most widely used Type II CRISPR system is derived from S. pyogenes and the PAM sequence is NGG located on the immediate 3′ end of the gRNA recognition sequence. The PAM sequences of Type II CRISPR systems from exemplary bacterial species include: Streptococcus pyogenes (NGG), Neisseria meningitidis (NNNNGATT), Streptococcus thermophilus (NNAGAA) and Treponema denticola (NAAAAC).

The term “Cas9 nickase” refers to a modified version of the Cas9-gRNA complex, as described above, containing a single inactive catalytic domain, i.e., either the RuvC- or the HNH-domain. With only one active nuclease domain, the Cas9 nickase cuts only one strand of the target DNA, creating a single-strand break or “nick”. A Cas9 nickase is still able to bind DNA based on gRNA specificity, though nickases will only cut one of the DNA strands. The majority of CRISPR plasmids currently being used are derived from S. pyogenes and the RuvC domain can be inactivated by an amino acid substitution at position D10 (e.g., D10A) and the HNH domain can be inactivated by an amino acid substitution at position H840 (e.g., H840A), or at positions corresponding to those amino acids in other proteins. As is known, the D10 and H840 variants of Cas9 cleave a Cas9-induced bubble at specific sites on opposite strands of the DNA. Depending on which mutant is used, the guide RNA-hybridized strand or the nonhybridized strand can be cleaved.

The following references are explicitly incorporated by reference for their teachings on Cas9, gRNA, and other reagents that can be used herein: Gasiunas et al. (Proc. Natl. Acad. Sci. 2012 109: E2579-E2586), Karvelis et al. (Biochem. Soc. Trans. 2013 41:1401-6), Pattanayak et al. (Nat. Biotechnol. 2013 31: 839-43), Jinek et al. (Elife 2013 2: e00471), Jiang et al. (Nat. Biotechnol. 2013 31:233-9), Hwang et al. (Nat. Biotechnol. 2013 31: 227-9), Mali et al. (Science 2013 339:823-6), Cong et al. (Science. 2013 339: 819-23), DiCarlo et al. (Nucleic Acids Res. 2013 41: 4336-43) and Qi et al. (Cell. 2013 152: 1173-83).

In some aspects, the present disclosure provides methods for enriching a target nucleic acid molecule using an endonuclease system derived from a CRISPR-Cas system. The present disclosure is based, in part, on the capability of CRISPR-Cas system to specifically bind with a target nucleic acid. Such target specific binding by the CRISPR-Cas system provides methods for efficiently enriching target nucleic acids, e.g., by pulling down an element of CRISPR-Cas that is associated with the target nucleic acids. CRISPR-Cas mediated nucleic acid enrichment bypasses traditionally required step of generating single-stranded nucleic acid prior to target specific binding, and enables directly targeting double-stranded nucleic acids, e.g., double-stranded DNA (dsDNA). In addition, CRISPR-Cas mediated nucleic acid binding is enzyme-driven, and thus it can offer faster kinetics and easier workflows for enrichment with lower temperature and/or isothermal reaction conditions.

In some embodiments, the method provided herein further includes separating the target nucleic acid molecule from the complex. In some embodiments, the CRISPR-Cas system can be bound to a surface, e.g., in plate once it has found the targeted region. This can prevent dissociation of the complex pre-maturely, and thus improve efficiency of capture. In some embodiments, the method provided herein further includes amplifying the target nucleic acid sequence.

In some embodiments, the target nucleic acid molecule provided herein is a double-stranded DNA (dsDNA). Certain CRISPR-Cas systems, e.g., Type II CRISPR-Cas systems, bind to double-stranded DNA in an enzyme-driven and sequence-specific manner. Therefore, one advantage provided herein is directly targeting double-stranded DNA, rather than processed single-stranded DNA, for enrichment.

In some embodiments, the endonuclease system provided herein is a Type I CRISPR-Cas system or a derivative thereof. In some embodiments, the endonuclease system provided herein is a Type II CRISPR-Cas system. In some embodiments, the endonuclease system provided herein is a Type III CRISPR-Cas system or a derivative thereof. The CRISPR-Cas systems provided herein include engineered and/or programmed nuclease systems derived from naturally occurring CRISPR-Cas systems. CRISPR-Cas systems can include contain engineered and/or mutated Cas proteins. CRISPR-Cas systems can also contain engineered and/or programmed guide RNA. For example, in some embodiments, crRNA and tracrRNA are synthesized by in vitro transcription, using a synthetic double stranded DNA template containing the T7 promoter. The tracrRNA has a fixed sequence, whereas the target sequence dictates part of crRNA's sequence. Equal molarities of crRNA and tracrRNA can be mixed and heated at 55° C. for 30 seconds. Cas9 can be added at the same molarity at 37° C. and incubated for 10 minutes with the RNA mix. 10-20 fold molar excess of Cas9 complex can be then added to the target DNA. The cleavage/binding reaction can occur within 15 minutes.

A target nucleic acid can be separated by pulling down its associated CRISPR-Cas system. In some embodiments, the endonuclease system is labeled, and the enzyme-nucleic acid complex is pulled down through the affinity label. In some embodiments, the crRNA or the derivative thereof is labeled. In some embodiments, the crRNA is labeled with biotin, as described above. In other embodiments, the tracrRNA is labeled as described above. In other embodiments, the Cas protein or the variant thereof is labeled with a capture tag. The protein capture tag includes, but not limited to, GST, Myc, hemagglutinin (HA), Green fluorescent protein (GFP), flag, His tag, TAP tag, and Fc tag. Other protein capture tags, e.g., affinity tags, recognized in the art can also be used in the present methods. Those skilled in the art will recognize that a protocol chosen for the purification step will be specific to the tag used. In some embodiments, anti-Cas protein antibodies or fragments thereof, e.g., anti-Cas9 antibodies, can also be used to separate the complex.

The key elements of a CRISPR-Cas system include a guide RNA, e.g., a crRNA, and a Cas protein. The crRNA or the derivative thereof contains a target specific nucleotide region complementary or substantially complementary to a region of the target nucleic acid. In some embodiments, the crRNA or the derivative thereof contains a user-selectable RNA sequence that permits specific targeting of the enzyme to a complementary double-stranded DNA. In some embodiments, the user-selectable RNA sequence contains 20-50 nucleotides complementary or substantially complementary to a region of the target DNA sequence. In some embodiments, the target specific nucleotide region of the crRNA has 100% base pair matching with the region of the target nucleic acid. In some embodiments, the target specific nucleotide region of the crRNA has 90%-100%, 80%-100%, or 70%-100% base pair matching with the region of the target nucleic acid. In some embodiments, there is one base pair mismatch between the target specific nucleotide region of the crRNA and the region of the target nucleic acid. In some embodiments, there are two base pair mismatches between the target specific nucleotide region of the crRNA and the region of the target nucleic acid. In some embodiments, there are three base pair mismatches between the target specific nucleotide region of the crRNA and the region of the target nucleic acid. In some embodiments, there are four base pair mismatches between the target specific nucleotide region of the crRNA and the region of the target nucleic acid. In some embodiments, there are five base pair mismatches between the target specific nucleotide region of the crRNA and the region of the target nucleic acid.

In some embodiments, the Cas9 protein or the variant thereof retains the two nuclease domains and is able to cleave opposite DNA strands and produce a double-stranded DNA break. In other embodiments, the Cas9 protein or the variant thereof is a Cas9 nickase and is able to produce a single-stranded nucleic acid nick, e.g., a single-stranded DNA nick. In some embodiments, only RuvC-nuclease domain is mutated and inactivated. In some embodiments, only HNH-nuclease domain is mutated and inactivated. In some embodiments, the Cas9 protein contains one inactivated nuclease domain having a mutation in the domain that cleaves a target nucleic acid strand that is complementary to the crRNA. In some embodiments, the mutation is D10A. In some embodiments, the Cas9 protein contains one inactivated nuclease domain having a mutation in the domain that cleaves a target nucleic acid strand that is noncomplementary to the crRNA. In some embodiments, the mutation is mutation is H840A. In yet other embodiments, the Cas9 protein or the variant thereof is a nuclease-null variant of the Cas9 protein, in which both RuvC- and HNH-active sites/nuclease domains are mutated. A nuclease-null variant of the Cas9 protein binds to double-stranded DNA, but not cleave the DNA, and thus it can be used for target specific DNA enrichment too. In some embodiments, the Cas9 protein has two inactivated nuclease domains with a first mutation in the domain that cleaves the strand complementary to the crRNA and a second mutation in the domain that cleaves the strand noncomplementary to the crRNA. In some embodiments, the Cas9 protein has a first mutation D10A and a second mutation H840A.

A CRISPR-Cas system can contain a Cas9 nickase in which one of the two nuclease domains is inactivated, e.g., D10A and H840 Cas9 mutants. The CRISPR-Cas system also contains a guide RNA, e.g., crRNA and crRNA-tracrRNA chimera, that contains a sequence substantially complementary to the target DNA sequence. The enzyme system binds to the target double-stranded DNA and creates a single-stranded nick. This nick serves as the starting point for nick translation using a nick translation polymerase, such as Bst. During the nick translation, biotinylated dNTPs are used to generate biotin labeled DNA fragment, so that the target DNA can be separated by adding magnetic streptavidin beads. In some embodiments, to prevent nonspecific nick translation, nicks present in the DNA prior to Cas9 cleavage can be removed using various methods known in the art, e.g., using DNA ligase, and 3′ and 5′ overhangs can also be filled in or chewed back with polymerase. In some embodiments, target nucleic acid molecules can first be treated with a cocktail of DNA polymerase, ligases and kinase to remove any preexisting nicks and recessive ends. Repaired DNA is incubated with Cas9 nickase complexes introducing single stranded nicks at targeted regions of the genome, which are used in nick translation reaction with biotinylated nucleotide. Biotinylated targeted regions of the genome are enriched with streptavidin coated beads in a pull down assay.

Further details of enrichment using CRISPR/Cas are provided in Examples 1-3 below.

### Enrichment Using Cas9-Transposase System

Disclosed herein are methods of enriching for target nucleic acid molecules, comprising (a) binding target nucleic acid molecules in a sample with a first Cas protein/gRNA complex that is specific to a first locus of a target region of the target nucleic acid molecules; (b) separating the target nucleic acid molecules of (a) from nontarget nucleic acid molecules in the sample; and (c) binding the separated target nucleic acid molecules of (b) with a second Cas protein/gRNA complex that is specific to a second locus of the target region of the target nucleic acid molecules, wherein a transposase is tethered to the first or second Cas protein/gRNA complex and the tethered transposase inserts a transposon end sequence tag in or near the binding site of the complex.

In some embodiments, the transposase is tethered to the first Cas protein/gRNA complex and the tethered transposase inserts a transposon end sequence tag near the binding site of the complex and the second Cas protein/gRNA complex comprises an inactive Cas protein. In some embodiments, the first Cas protein/gRNA complex comprises an inactive Cas protein and the transposase is tethered to the second Cas protein/gRNA complex and the tethered transposase inserts a transposon end sequence tag near the binding site of the complex. In some embodiments, the transposase is tethered to the first Cas protein/gRNA complex and to the second Cas protein/gRNA complex and the tethered transposases insert a transposon end sequence tag near the binding sites of the complexes.

In some embodiments, the transposase tethered to the first or second Cas protein/gRNA complex can be a dCas9-Tn5 fusion protein. The transposon end sequence tag can be attached to an affinity label.

The methods can further comprise pulling down tagmented nucleic acid molecules with an affinity label partner. The methods can further comprise washing the pulled down tagmented nucleic acid molecules. The methods can further comprise binding anti-dCas9 beads to capture the target nucleic acid molecules. The methods can further comprise eluting the bound nucleic acid molecule off the beads.

A fusion protein comprising a Cas9 protein and a transposase is provided herein.

In some embodiments, the Cas9 protein has inactivated nuclease activity.

In some embodiments, the Cas9 protein is fused to the N-terminus of the transposase.

In some embodiments, the Cas9 protein is fused to the C-terminus of the transposase.

A complex comprising a fusion protein comprising a Cas9 protein and a transposase is provided herein, where the complex further comprises a Cas9-associated guide RNA and a transposon.

In some embodiments, Cas9 protein and Cas9-associated guide RNA directs the transposon to a defined site in a genome, thereby allowing the transposase to insert the transposon at a defined site.

In some embodiments, the transposon comprises one or more primer binding sites, a molecular barcode, or a promoter.

Also provided are methods comprising contacting the complex with a genome, thereby causing the transposon to be inserted into the genome proximal at a site to which the Cas9 protein binds.

In some embodiments, the methods can be performed by contacting a plurality of complexes with a genome, wherein each complex comprises a different guide RNA, and the different guide RNAs are complementary to defined sites in the genome, and inserting a plurality of transposons into the genome.

In some embodiments, the sequences between the transposon insertions are amplified using PCR primers that bind to primer binding sites in the transposon insertions.

In some embodiments, the transposon is biotinylated.

In some embodiments, the transposase is a Sleeping Beauty, Piggybac or Tn5 transposase.

Transposases are enzymes derived from transposons that randomly break DNA and insert a transposon DNA that encodes the transposase. Transposases have been used in genetic and molecular biology applications to rapidly integrate DNA “tags” into a target sample of DNA (usually genomic DNA) as part of an insertional mutagenesis screen (in vivo) or more recently to create next-generation sequencing libraries (in vitro).

As with transposon integration, the integration of DNA tags show little sequence bias except insertion between TA dinucleotides (which are duplicated during transposition and flank the integration site). For some next generation sequencing (NGS) applications, whole-genome surveys are benefitted by the random integration events garnered from transposition, which is the basis of the Nextera whole-genome library preparation technology from Illumina. However, for creating targeted NGS libraries, it would be advantageous to target a transposase to specific genomic locations to enable the rapid production of “directed” NGS libraries. The types of “targeted” NGS libraries envisioned here would obviate the hybridization-based selection approaches used in target capture protocols, as these selections take extra time, and could permit time-sensitive applications (such as diagnostics).

As used herein, the term “tagmentation,” “tagment,” or “tagmenting” refers to transforming a nucleic acid, e.g., a DNA, into adaptor-modified templates in solution ready for cluster formation and sequencing by the use of transposase mediated tagging. This process often involves the modification of the nucleic acid by a transposome complex comprising transposase enzyme that can insert a transposon end sequence tag into the target nucleic acid molecule. Tagmentation results in the simultaneous fragmentation of the nucleic acid and ligation of the adaptors to the 5′ ends of both strands of duplex fragments. Following a purification step to remove the transposase enzyme, additional sequences can be added to the ends of the adapted fragments by PCR.

As used herein, the term “transposome complex” refers to a transposase enzyme non-covalently bound to a double stranded nucleic acid. For example, the complex can be a transposase enzyme preincubated with double-stranded transposon DNA under conditions that support noncovalent complex formation. Double-stranded transposon DNA can include, without limitation, Tn5 DNA, a portion of Tn5 DNA, a transposon end composition, a mixture of transposon end compositions or other double-stranded DNAs capable of interacting with a transposase such as the hyperactive Tn5 transposase.

A “transposase” means an enzyme that is capable of forming a functional complex with a transposon end-containing composition (e.g., transposons, transposon ends, transposon end compositions) and catalyzing insertion or transposition of the transposon end-containing composition into the double-stranded target nucleic acid with which it is incubated, for example, in an in vitro transposition reaction. A transposase as presented herein can also include integrases from retrotransposons and retroviruses. Transposases, transposomes and transposome complexes are generally known to those of skill in the art, as exemplified by the disclosure of US 2010/0120098, the content of which is incorporated herein by reference in its entirety. Although many embodiments described herein refer to Tn5 transposase and/or hyperactive Tn5 transposase, it will be appreciated that any transposition system that is capable of inserting a transposon end with sufficient efficiency for its intended purpose can be used in the present disclosure. In particular embodiments, a transposition system is capable of inserting the transposon end in a random or in an almost random manner to or near the target region of the target nucleic acid.

As used herein, a “tethered transposase” is a transposase that is covalently or noncovalently associated with another protein or nucleic acid molecule.

As used herein, the term “transposition reaction” refers to a reaction wherein one or more transposons are inserted into target nucleic acids, e.g., at random sites or almost random sites. Essential components in a transposition reaction are a transposase and DNA oligonucleotides that exhibit the nucleotide sequences of a transposon, including the transferred transposon sequence and its complement (the nontransferred transposon end sequence) as well as other components needed to form a functional transposition or transposome complex. The DNA oligonucleotides can further comprise additional sequences (e.g., adaptor or primer sequences) as needed or desired. In some embodiments, the method provided herein is exemplified by employing a transposition complex formed by a hyperactive Tn5 transposase and a Tn5-type transposon end (Goryshin and Reznikoff, 1998, J. Biol. Chem., 273: 7367) or by a MuA transposase and a Mu transposon end comprising R1 and R2 end sequences (Mizuuchi, 1983, Cell, 35: 785; Savilahti et al., 1995, EMBO J., 14: 4893). However, any transposition system that is capable of inserting a transposon end in a random or in an almost random manner with sufficient efficiency to 5′-tag and fragment a target DNA for its intended purpose can be used in the present disclosure. Examples of transposition systems known in the art which can be used for the present methods include but are not limited to Staphylococcus aureus Tn552 (Colegio et al., 2001, J Bacterid., 183: 2384-8; Kirby et al., 2002, Mol Microbiol, 43: 173-86), TyI (Devine and Boeke, 1994, Nucleic Acids Res., 22: 3765-72 and International Patent Application No. WO 95/23875), Transposon Tn7 (Craig, 1996, Science. 271: 1512; Craig, 1996, Review in: Curr Top Microbiol Immunol, 204: 27-48), TnIO and ISlO (Kleckner et al., 1996, Curr Top Microbiol Immunol, 204: 49-82), Mariner transposase (Lampe et al., 1996, EMBO J., 15: 5470-9), Tci (Plasterk, 1996, Curr Top Microbiol Immunol, 204: 125-43), P Element (Gloor, 2004, Methods Mol Biol, 260: 97-114), TnJ (Ichikawa and Ohtsubo, 1990, J Biol Chem. 265: 18829-32), bacterial insertion sequences (Ohtsubo and Sekine, 1996, Curr. Top. Microbiol. Immunol. 204:1-26), retroviruses (Brown et al., 1989, Proc Natl Acad Sci USA, 86: 2525-9), and retrotransposon of yeast (Boeke and Corces, 1989, Annu Rev Microbiol. 43: 403-34). The method for inserting a transposon end into a target sequence can be carried out in vitro using any suitable transposon system for which a suitable in vitro transposition system is available or that can be developed based on knowledge in the art. In general, a suitable in vitro transposition system for use in the methods provided herein requires, at a minimum, a transposase enzyme of sufficient purity, sufficient concentration, and sufficient in vitro transposition activity and a transposon end with which the transposase forms a functional complex with the respective transposase that is capable of catalyzing the transposition reaction. Suitable transposase transposon end sequences that can be used in the methods disclosed herein include but are not limited to wild-type, derivative or mutant transposon end sequences that form a complex with a transposase chosen from among a wild-type, derivative or mutant form of the transposase.

The term “transposon end” (TE) refers to a double-stranded nucleic acid, e.g., a double-stranded DNA, that exhibits only the nucleotide sequences (the “transposon end sequences”) that are necessary to form the complex with the transposase or integrase enzyme that is functional in an in vitro transposition reaction. In some embodiments, a transposon end sequence tag is capable of forming a functional complex with the transposase in a transposition reaction. As nonlimiting examples, transposon ends can include the 19-bp outer end (“OE”) transposon end, inner end (“IE”) transposon end, or “mosaic end” (“ME”) transposon end recognized by a wild-type or mutant Tn5 transposase, or the R1 and R2 transposon end as set forth in the disclosure of US 2010/0120098, the content of which is incorporated herein by reference in its entirety. Transposon ends can include any nucleic acid or nucleic acid analogue suitable for forming a functional complex with the transposase or integrase enzyme in an in vitro transposition reaction. For example, the transposon end can include DNA, RNA, modified bases, nonnatural bases, modified backbone, and can include nicks in one or both strands. Although the term “DNA” is sometimes used in the present disclosure in connection with the composition of transposon ends, it should be understood that any suitable nucleic acid or nucleic acid analogue can be utilized in a transposon end.

In some embodiments, the endonuclease system provided herein further includes a transposase, and thus transposase is part of the endonuclease system, and the method of the present disclosure further includes adding transposon end to the target DNA sequence; and tagmenting the target DNA sequence by the transposase. In some embodiments, the transposase binds to a nucleotide sequence of the endonuclease system. In some embodiments, the transposase binds to a crRNA or a derivative thereof. In some embodiments, the transposase binds to a tracrRNA or a derivative thereof. In some embodiments, the transposase binds to a sgRNA or a chimeric polynucleotide having a crRNA polynucleotide and a tracrRNA polynucleotide. In some embodiments, the transposon end is a mosaic end (ME), and the transposase is a Tn5 transposase. In some embodiments, a transposase (Tn5) binds to the endonuclease system through an aptamer connected to the crRNA-tracrRNA chimera. Thus, Tn5 binds to the system without the assistance of ME sequences. The endonuclease system containing Tn5 is added and binds to the target DNA. ME sequences is then added to the DNA, and thus the DNA can be tagmented by Tn5. In other embodiments, the transposase provided herein and the Cas protein provided herein form a fusion protein. The endonuclease system containing Tn5 is added and binds to the target DNA. ME sequences is then added to the DNA, and thus the DNA can be tagmented by Tn5 and sequences, e.g., index or universal primer sequences, can be introduced.

In some embodiments, a Tn5 system and a CRISPR-Cas9 system are added to a population of nucleic acid containing a target nucleic acid molecule(s). CRISPR-Cas9 system contains a Cas9 with two nuclease domains. Thus, both the Tn5 system and the CRISPR-Cas9 system can cut nucleic acid, and after the cutting, both systems are staying with the cleaved ends of nucleic acid. The CRISPR-Cas9 system is labeled, through which the target nucleic acid can be pulled down. After treated with proteases, the DNA fragments generated from the target nucleic acid are released and can be subject to further amplification and/or library preparation. Further details using Cas9-Transposase are provided in Example 4 below.

### Methods for Negative and Positive Enrichment/Selection of Target Nucleic Acid Molecules

In some embodiments, provided methods and compositions take advantage of a targeted endonuclease (e.g., a ribonucleoprotein complex (CRISPR-associated endonuclease such as active or inactive Cas9, Cpfl), a homing endonuclease, a zinc-fingered nuclease, a TALEN, an argonaute nuclease, and/or a meganuclease (e.g., megaTAL nuclease, etc.), or combinations thereof) or other technology capable of site-directed interaction with nucleic acid molecules, to positively enrich for desired (on-target) nucleic acid molecules. Other embodiments provide methods and such compositions to negatively enrich/select for desired nucleic acid molecules by way of removing undesired (e.g., off-target) nucleic acid molecules from the sample. Some embodiments described herein combine both positive and negative enrichment schemes. In some embodiments, analyzing can be or comprise quantitation and/or sequencing.

The enriched DNA fragments can be ligated to adapters for nucleic acid quantification or analysis, such sequencing. For example, the blunt ends of the target fragment can be directly ligated to blunt-ended adapters. Aspects of ligating adapters to the cleaved double-stranded nucleic acid molecules can include end-repair and 3′-dA-tailing of the fragments, if required in a particular application. In other embodiments, further processing of the fragments to generate suitable ligateable ends of the fragment can include can be any of a variety of forms or steps to form a ligateable end having, for example, a blunt end, an A-3′ overhang, a “sticky” end comprising a one nucleotide 3′ overhang, a two nucleotide 3′ overhang, a three nucleotide 3′overhang, a 4, 5, 6, 7, 8, 9, 10, 11, 12, 13, 14, 15, 16, 17, 18, 19, 20 or more nucleotide 3′ overhang, a one nucleotide 5′ overhang, a two nucleotide 5′ overhang, a three nucleotide 5′ overhang, a 4, 5, 6, 7, 8, 9, 10, 11, 12, 13, 14, 15, 16, 17, 18, 19, 20 or more nucleotide 5′ overhang, among others. The 5′ base of the ligation site can be phosphorylated and the 3′ base can have a hydroxyl group, or either can be, alone or in combination, dephosphorylated or dehydrated or further chemically modified to either facilitate enhanced ligation of one strand to prevent ligation of one strand, optionally, until a later time point.

In another embodiment, positive enrichment/selection of target nucleic acid molecules using CRISPR/Cas can be facilitated by affinity-based enrichment of target nucleic acid molecules. A CRISPR/Cas9 ribonucleoprotein complex can comprises an affinity label (e.g., biotin). The affinity label can be incorporated on the gRNA (e.g., crRNA, tracrRNA) or on the Cas9 protein. Accordingly, the ribonucleoprotein complex provides an affinity label for later pull-down steps.

Guide RNA (gRNA)-facilitated binding of the variant Cas9 ribonucleoprotein complex presenting the affinity label is followed by cleavage of the double-stranded target DNA. Following cleavage and while Cas9 remains bound to the cleaved 5′ and 3 ends of the target DNA fragment, the reaction mixture is brought into contact with a functionalized surface with one or more extraction moieties bound thereto. The provided extraction moieties are capable of binding to the affinity label (e.g., a streptavidin bead where the affinity label is biotin) for immobilization and separation of molecules bearing the affinity label. In particular, the extraction moiety can be any member of a binding pair, such as biotin/streptavidin or hapten/antibody or complementary nucleic acid sequences (DNA/DNA pair, DNA/RNA pair, RNA/RNA pair, LNA/DNA pair, etc.). For example, an affinity label that is attached to a CRISPR/Cas9 ribonucleoprotein complex that is bound to a (cleaved) target dsDNA fragment is captured by its binding pair (e.g., the extraction moiety) which is attached to an isolatable moiety (e.g., such as a magnetically attractable particle or a large particle that can be sedimented through centrifugation). Accordingly, the affinity label can be any type of molecule/moiety that allows affinity separation of nucleic acids associated with (e.g., bound by Cas9) the affinity label from nucleic acids lacking association with the affinity label. An example of an affinity label is biotin which allows affinity separation by binding to streptavidin linked or linkable to a solid phase or an oligonucleotide, which in turn allows affinity separation through binding to a complementary oligonucleotide linked or linkable to a solid phase. Undesired or nontargeted nucleic acid molecules can remain free in solution. Beneficially, free/unbound nucleic acid molecules, which does not bear or is associated with any affinity label, can be effectively removed/separated from the desired target nucleic acid molecules. In further embodiments, the functionalized surface (S) can be washed to remove residual byproducts or other contaminants.

Undesired or nontarget nucleic acid molecules can be substantially reduced in abundance. Collection of the desired/target nucleic acid fragments can be accomplished in any application-appropriate manner. By way of specific example, in some embodiments, collection of desired nucleic acid molecules can be accomplished via one or more of removal of the functionalized surface via size filtration, magnetic methods, electrical charge methods, centrifugation density methods or any other methods or, collection of elution fractions if using column-based purification methods or similar, or by any other commonly understood purification practice by one experienced in the art. In addition to use of targeted endonuclease(s), any other application appropriate method(s) of achieving nucleic acid molecules of a substantially uniform length can be used. By way of nonlimiting example, such methods can be or include use of one or more of: an agarose or other gel, gel electrophoresis, an affinity column, HPLC, PAGE, filtration, gel filtration, exchange chromatography, SPRI/Ampure type beads, or any other appropriate method as will be recognized by one of skill in the art.

In some embodiments, the affinity-based positive enrichment steps can be combined or used in conjunction with negative enrichment steps. For example, following cleavage and while Cas9 remains bound to the cleaved 5′ and 3 ends of the target DNA fragment (either before or after the affinity-based enrichment step), the sample can be treated with an exonuclease to destroy any unwanted nucleic acid molecules or contaminants in the sample. After the affinity-based enrichment step and optional negative exonuclease clean up steps, Cas9 is disassociated from the DNA to release a blunt-ended double-stranded target DNA fragment. Optionally, the above enrichment steps can be combined with a size-based enrichment step as described above, and in some embodiments, the enriched DNA fragments can be ligated to adapters for nucleic acid interrogation, such sequencing as discussed above.

Exonuclease resistant adapter-nucleic acid complexes can be further enriched via size selection or via target sequence (e.g., CRISPR/Cas9 pull-down). In other embodiments, the hairpin adapters bearing an affinity label can used, which are directly suitable for affinity-based enrichment using functionalized surfaces with exposed extraction moieties.

Elution of the targeted fragments can occur via release from the extraction moieties. In some nonlimiting examples, a cleavable moiety can be incorporated proximate the bound end of the oligonucleotide extraction moiety. In other embodiments, temperature or other conditions can be changed to cause denaturing of the short affinity label/extraction binding while maintaining the double-stranded nature of the target nucleic acid fragment. In still other embodiments, hairpin adapters can be used at a second sticky end of the target fragments to tether the duplex strands together during elution and further processing. In various embodiments, after enrichment steps, the sticky ends can be polished, trimmed or biocomputationally filtered as described herein for avoiding pseudoplex errors.

As used herein, the term “detecting” a nucleic acid molecule or fragment thereof refers to determining the presence of the nucleic acid molecule, typically when the nucleic acid molecule or fragment thereof has been fully or partially separated from other components of a sample or composition, and also can include determining the charge-to-mass ratio, the mass, the amount, the absorbance, the fluorescence, or other property of the nucleic acid molecule or fragment thereof.

The present disclosure, among other things, provides methods and reagents for affinity-based enrichment of target nucleic acid molecules. In some embodiments including such methods, one or more affinity labels or moieties can be used for enrichment/selection of desired target nucleic acid molecules from samples comprising genomic material, off-target nucleic acid molecules, contaminating nucleic acid molecules, nucleic acid molecules from mixed samples, cfDNA material, etc. For example, some embodiments comprise use of one or more affinity labels/moieties for positive enrichment/selection of desired target nucleic acid molecules (e.g., fragments comprising target sequence or genomic regions of interest, targeted genomic regions of interest within unfragmented genomic DNA). In other embodiments, affinity labels can be used for negative enrichment/selection to exclude or reduce the abundance of nondesired genomic material.

For example, in some embodiments including positive enrichment, an adapter oligonucleotide can have an affinity label that is or comprises an affixed chemical moiety (e.g., biotin) that can be used to isolate or separate desired adapter-nucleic acid complexes via capture in one or more subsequent purification steps, for example, via an extraction moiety (e.g., streptavidin) bound to a functionalized surface (e.g. a paramagnetic bead or other form of bead). In some embodiments including negative enrichment, an affinity label that is or comprises an affixed chemical moiety (e.g., biotin) can be used to purify out or separate undesired genomic material ligated or attached to an adapter (or other probe comprising the affinity label) (e.g., off-target nucleic acid fragments, etc.) via capture in one or more subsequent purification steps, for example, via an extraction moiety (e.g., streptavidin) bound to a functionalized surface (e.g., a paramagnetic bead or other form of bead).

### Separation Methods

As is described herein, various methods include at least one separation step. It is specifically contemplated that any of a variety of separation steps can be included in various embodiments. For example, in some embodiments, separation can be or comprise physical separation, virtual separation, size separation, magnetic separation, solubility separation, charge separation, hydrophobicity separation, polarity separation, electrophoretic mobility separation, density separation, chemical elution separation, SBIR bead separation etc. For example, a physical group can have a magnetic property, a charge property, or an insolubility property. In embodiments, when the physical group has a magnetic property and a magnetic field is applied, the associated adapter nucleic acid sequences including the physical group is separated from the adapter nucleic acid sequences not including the physical group. In other embodiments, when the physical group has a charge property and an electric field is applied, the associated adapter nucleic acid sequences including the physical group is separated from the adapter nucleic acid sequence not including the physical group. In embodiments, when the physical group has an insolubility property and the adapter nucleic acid sequences are contained in a solution for which the physical group is insoluble, the adapter nucleic acid sequences comprising the physical group is precipitated away from the adapter nucleic acid sequence not including the physical group which remains in solution.

Any of a variety of physical separation methods can be included in various embodiments. By way of specific example, a nonlimiting set of physical separation methods includes size selective filtration, density centrifugation, HPLC separation, gel filtration separation, FPLC separation, density gradient centrifugation and gel chromatography, among others.

Any of a variety of magnetic separation methods can be included in various embodiments. Typically, magnetic separation methods will encompass the inclusion or addition of one or more physical groups having a magnetic property such that, when a magnetic field is applied, molecules including such physical group(s) are separated from those that do not. By way of specific example, physical groups that include exhibit a magnetic property include, but are not limited to ferromagnetic materials such as iron, nickel, cobalt, dysprosium, gadolinium and alloys thereof. Commonly used paramagnetic beads for chemical and biochemical separation embed such materials within a surface that reduces chemical interaction of the materials with the chemicals being manipulated, such as polystyrene, which can be functionalized for the affinity properties discussed above.

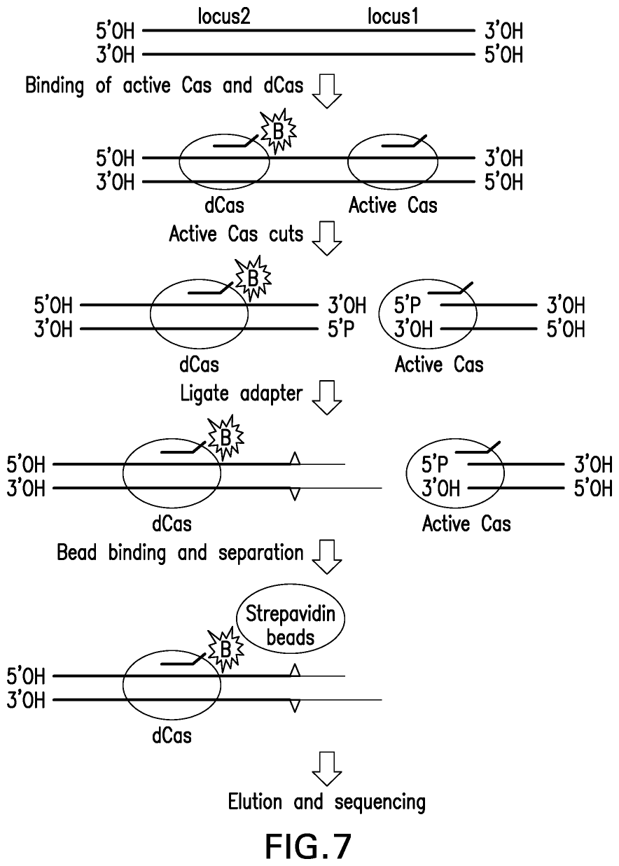

“Virtual separation” allows separation of target nucleic acid molecules and nontarget nucleic acid molecules without a need for physical separation. For example, a first Cas protein cut and adapter ligation “virtually separate” target nucleic acid molecules from nontarget nucleic acid molecules substantially. If the resulting adapter ligated DNA were to be sequenced directly, it would have a low enrichment factor. A second separation using dCas protein/gRNA complex bound on the second locus will add further specificity to the system, because nontarget nucleic acid molecules accidentally ligated to the adapter would not be bound by second dCas protein/gRNA complex, and nontarget nucleic acid molecules accidentally bound by second dCas protein/gRNA complex would not be ligated to the adapter at the first cut locus. Thus, the first “virtual separation” can be ligating an adapter to either blunt ends (e.g. generated by Cas9) or sticky ends (e.g. generated by Cas12a) at the cut site, in combination with or without pre-treating DNA to block native fragment ends from ligation. The first and second sites and their association with either active Cas or dCas protein/gRNA complex can also be reversed to what is illustrated in FIG. 7.

### Affinity or Capture Labels

As is described herein, in some embodiments, an affinity label can be present in any of a variety of configurations on proteins, along oligonucleotide probes, adapters, ribonucleotide sequences, ribonucleoprotein complexes, etc. In some embodiments, an affinity label can be incorporated or affixed to an oligonucleotide or adapter strand in a region 5′ of the sequence. In some embodiments, an affinity label can be present somewhere in the middle of an oligonucleotide strand (i.e., not on the 5′ or 3′ end of the oligonucleotide). In embodiments including two or more affinity labels, each affinity label can be present at a different location along the oligonucleotides or adapter(s).

As used herein, the term “affinity label” or “capture label” (which can also be referred to as a “capture tag”, “capture moiety”, “affinity tag”, “epitope tag”, “tag”, “prey” moiety or chemical group, among other names) refers to a moiety that can be integrated into, or onto, a target molecule, or substrate, for the purposes of purification. In some embodiments, the affinity label is selected from a group comprising a small molecule, a nucleic acid, a peptide, or any uniquely bindable moiety. In some embodiments, the affinity label is affixed to the 5′ of a nucleic acid molecule. In some embodiments, the affinity label is affixed to the 3′ of a nucleic acid molecule. In some embodiments, the affinity label is conjugated to a nucleotide within the internal sequence of a nucleic acid molecule not at either end. In some embodiments, the affinity label is a sequence of nucleotides within the nucleic acid molecule. In some embodiments, the affinity label can be biotin, biotin deoxythymidine dT, biotin NHS, biotin TEG, desthiobiotin NHS, digoxigenin NHS, DNP TEG, or thiols, among others. In some embodiments, affinity labels include, without limitation, biotin, avidin, streptavidin, a hapten recognized by an antibody, a particular nucleic acid sequence, and magnetically attractable particles. In some embodiments, chemical modification (e.g., Acridite™-modified, adenylated, azide-modified, alkyne-modified, I-Linker™-modified etc.) of nucleic acid molecules can serve as an affinity label.

In some embodiments, an affinity label is selected from a group of biotin, biotin deoxythymidine dT, biotin NHS, biotin TEG, Biotin-6-Aminoaliyl-2′-deoxyuridine-S′-Triphosphate, Biotin-16-Aminoallyl-2-deoxycytidine-5′-Triphosphate, Biotinyl 6-Aminoally lcytidine-5′-Triphosphate, N4-Biotin-OBEA-2′-deoxycytidine-5′-Triphosphate, Biotin-16-Aminoallyluridine-5′-Triphosphate, Biotin-16-7-Deaza-7-Aminoally 1-2′-deoxygnanosine-5′-Triphosphate, 5′-Biotin-G-Monophosphate, 5′-Biotin-A-Monophosphate, 5′-Biotin-dG-Monophosphate, 5′-Biotin-dA-Monophosphate, desthiobiotin NHS, Desthiobiotin-6-Aminoallyl-2′-deoxycytidine-5′-Triphosphate, digoxigenin NHS, DNP TEG, thiols, Colicin E2, Im2, glutathione, glutathione-s-transferase (GST), nickel, polyhistidine, FLAG-tag, myc-tag, among others. In some embodiments, affinity labels include, without limitation, biotin, avidin, streptavidin, a hapten recognized by an antibody, a particular nucleic acid sequence and/or magnetically attractable particle. In some embodiments, one or more chemical modifications of nucleic acid molecules (e.g., Acridite™-modified among many other modifications, some of which are described elsewhere in the application) can serve as an affinity label.

### Affinity Label Partner or Extraction Moieties

As used herein the term “affinity label partner” or “extraction moiety” (which can also be referred to as a “binding partner”, an “affinity partner”, a “bait” moiety or chemical group among other names) refers to an isolatable moiety or any type of molecule that allows affinity separation of nucleic acids bearing the affinity label from nucleic acids lacking the affinity label. In some embodiments, the extraction moiety is selected from a group comprising a small molecule, a nucleic acid, a peptide, an antibody or any uniquely bindable moiety. The extraction moiety can be linked or linkable to a solid phase or other surface for forming a functionalized surface. In some embodiments, the extraction moiety is a sequence of nucleotides linked to a surface (e.g., a solid surface, bead, magnetic particle, etc.). In some embodiments, the extraction moiety is selected from a group of avidin, streptavidin, an antibody, a polyhistidine tag, a FLAG tag or any chemical modification of a surface for attachment chemistry. Nonlimiting examples of these latter include azide and alkyne groups which can form 1,2,3-triazole bonds via “Click” methods, or thiol an azide and terminal alkyne, thiol-modified surfaces can covalently react with Acrydite-modified oligonucleotides and aldehyde and ketone modified surfaces which can react to affix I-Linker™ labeled oligonucleotides.

Extraction moieties can be a physical binding partner or pair to targeted affinity label and refers to an isolatable moiety or any type of molecule that allows affinity separation of nucleic acids bearing the affinity label or bound by an affinity label bearing molecule (e.g., oligonucleotide, protein, ribonucleoprotein complex, etc.) from nucleic acids lacking the affinity label. Extraction moieties can be directly linked or indirectly linked (e.g., via nucleic acid, via antibody, via aptamer, etc.) to a substrate, such as a solid surface. In some embodiments, the extraction moiety is selected from a group comprising a small molecule, a nucleic acid, a peptide, an antibody or any uniquely bindable moiety. The extraction moiety can be linked or linkable to a solid phase or other surface for forming a functionalized surface. In some embodiments, the extraction moiety is a sequence of nucleotides linked to a surface (e.g., a solid surface, bead, magnetic particle, etc.). In some embodiments, wherein the affinity label is biotin, the extraction moiety is selected from a group of avidin or streptavidin. It will be appreciated by one of skill in the art, any of a variety of affinity binding pairs can be used in accordance with various embodiments.

In certain embodiments, extraction moieties can be physical or chemical properties that interact with the targeted affinity label. For example, an extraction moiety can be a magnetic field, a charge field or a liquid solution in which a targeted affinity label is insoluble. Such physical or chemical properties can be applied and adapter nucleic acids bearing the affinity label can be immobilized within/against a vessel (surface) or column. Depending on the desired positive enrichment/selection or negative enrichment/selection outcome, the immobilized molecules can be retained (positive enrichment) or the nonimmobilized molecules can be retained (negative enrichment) for further purification/processing or use.

By “specifically bind” is meant that the affinity label binds with specificity to an affinity partner on a solid support to differentiate between the pair and other components or contaminants of the system. The binding should be sufficient to remain bound under the conditions of the assay, including wash steps to remove nonspecific binding. In some embodiments, the dissociation constants of the pair will be less than about 10⁻⁴-10⁻⁶ M⁻¹, with less than about 10⁻⁵ to 10⁻⁹ M⁻¹, or less than about 10⁻⁷-10⁻⁹ M⁻¹.

Thus, the nucleic acid comprising the sample tag sequence can be immobilized on a solid surface or support rather than nonsample tag oligonucleotides.

The nonhybridized nucleic acids (nontarget nucleic acid molecules) can be removed by washing. For example, the hybridization complexes are immobilized on a solid support and washed under conditions sufficient to remove nonhybridized nucleic acids, i.e. nonhybridized probes and sample nucleic acids. In certain embodiments, immobilized complexes are washed under conditions sufficient to remove imperfectly hybridized complexes. That is, hybridization complexes that contain mismatches are also removed in the wash steps.

A variety of hybridization or washing conditions can be used, including high, moderate and low stringency conditions; see for example, Maniatis et al., Molecular Cloning: A Laboratory Manual, 2d Edition, 1989, and Short Protocols in Molecular Biology, ed. Ausubel, et al, hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength and pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of helix destabilizing agents such as formamide. The hybridization or washing conditions can also vary when a nonionic backbone, i.e., PNA is used, as is known in the art. In addition, cross-linking agents can be added after target binding to cross-link, i.e., covalently attach, the two strands of the hybridization complex.

In some embodiments, it is desirable to release or cleave the target nucleic acid molecules attached to the affinity labels.

In the methods disclosed herein, the releasing can comprise releasing the target nucleic acid molecules from the affinity labels by, but not limited to, heat or alkaline denaturation. The releasing can comprise releasing the target nucleic acid molecules from the affinity labels by uracil-DNA glycosylase digestion of the dU base in the adapter.

### Solid Surfaces

When the affinity partner/extraction moiety is attached to a solid surface or substrate and bound to the affinity label, the adapter nucleic acid sequences including the affinity label are capable of being separated from the adapter nucleic acid sequence not including the affinity label. A solid surface or substrate can be a bead, isolatable particle, magnetic particle or another fixed structure.

Where beads are used, it is not intended that the disclosure herein be limited to the particular type. A variety of bead types are commercially available, including but not limited to, beads selected from agarose beads, streptavidin-coated beads, NeutrAvidin-coated beads, antibody-coated beads, paramagnetic beads, magnetic beads, electrostatic beads, electrically conducting beads, fluorescently labeled beads, colloidal beads, glass beads, semiconductor beads, and polymeric beads.

As is described herein and will be appreciated by one of skill in the art, any of a variety of functionalized surfaces can be used in accordance with various embodiments. For example, in some embodiments, a functionalized surface can be or comprise a bead (e.g., a controlled pore glass bead, a macroporous polystyrene bead, etc.). However, it will be understood to one of skill in the art that many other chemical moiety/surface pairs could be similarly used to achieve the same purpose. It will be understood that the specific functionalized surfaces described here are meant only as examples, and that any other appropriate fixed structure or substrate capable of being associated with (e.g., linked to, bound to, etc.) one or more extraction moieties can be used.

As used herein, the term “functionalized surface” refers to a solid surface, a bead, or another fixed structure that is capable of binding or immobilizing an affinity label. In some embodiments, the functionalized surface comprises an extraction moiety capable of binding an affinity label. In some embodiments, an extraction moiety is linked directly to a surface. In some embodiments, chemical modification of the surface functions as an extraction moiety. In some embodiments, a functionalized surface can comprise controlled pore glass (CPG), magnetic porous glass (MPG), among other glass or nonglass surfaces. Chemical functionalization can entail ketone modification, aldehyde modification, thiol modification, azide modification, and alkyne modifications, among others. In some embodiments, the functionalized surface and an oligonucleotide used for adapter synthesis are linked using one or more of a group of immobilization chemistries that form amide bonds, alkylamine bonds, thiourea bonds, diazo bonds, hydrazine bonds, among other surface chemistries. In some embodiments, the functionalized surface and an oligonucleotide used for adapter synthesis are linked using one or more of a group of reagents including EDAC, NHS, sodium periodate, glutaraldehyde, pyridyl disulfides, nitrous acid, biotin, among other linking reagents.

### Compositions

In addition to the method described above, a number of compositions are also provided. In certain embodiments, the composition(s) can contain 1, 2 or more, 3 or more, 4 or more, 5 or more, or 10 or more Cas9-associated guide RNAs that are each complementary to a different, pre-defined, site in a genome. The composition can comprise, e.g., at least 10, at least 15, at least 20, at least 30, at least 50, at least 75, at least 100, at least 200, at least 300, at least 400, at least 500, at least 600, at least 700, at least 800, at least 900, at least 1,000, or at least 10,000 or more guide RNAs. The sites to which the Cas9-associated guide RNAs bind are immediately downstream from a PAM trinucleotide (e.g., CCN). The guide RNAs can be in solution, or they can be in dried form, e.g., lyophilized. The guide RNAs can be at least 20, at least 30, at least 50, at least 75, at least 100, at least 150, at least 180, at least 200, at least 220, at least 240, or at least 260 nucleotides long. Such compositions can be employed in any embodiments disclosed herein.

As would be apparent, the composition can additionally contain a single Cas9 protein. The composition can also contain genomic DNA, e.g., microbial or mammalian genomic DNA such as human genomic DNA.

The guide RNAs can be synthesized on a solid support in an array, where the oligonucleotides are grown in situ. Oligonucleotide arrays can be fabricated using any means, including drop deposition from pulse jets or from fluid-filled tips, etc., or using photolithographic means. Polynucleotide precursor units (such as nucleotide monomers), in the case of in situ fabrication can be deposited. Oligonucleotides synthesized on a solid support can then be cleaved off to generate the population of oligonucleotides. Such methods are described in detail in, for example U.S. Pat. Nos. 7,385,050, 6,222,030, and 6,323,043, and US2002/0058802, etc., the disclosures of which are incorporated herein by reference. The oligonucleotides can be tethered to a solid support via a cleavable linker and cleaved from the support before use.

In some embodiments, the Cas9-associated guide RNAs are each specific for a different, pre-defined, site in genomic DNA.

In some embodiments, the Cas9-associated guide RNAs are each specific for a different, pre-defined, site in mammalian genomic DNA.

In some embodiments, the Cas9-associated guide RNAs are each specific for a different, pre-defined, site in human genomic DNA.

In some embodiments, the Cas9-associated guide RNAs are each specific for a different, pre-defined, site in microbial genomic DNA.

In some embodiments, the composition comprises one or a plurality of Cas9-associated guide RNA binding to the genome of one pathogen and one or a plurality of Cas9-associated guide RNA binding to the genome of another pathogen.

In some embodiments, the composition further comprises a Cas9 nuclease.

In some embodiments, the Cas9-associated guide RNAs are in solution as a mixture.

In some embodiments, the Cas9-associated guide RNAs are tethered to a substrate in an array. In some embodiments, the composition comprises a DNase inhibitor.

### Kits

Also disclosed herein are kits for practicing the subject method, as described above. The subject kit contains mutant Cas9 protein and 1 or a set of at least 2, at least 5, at least 10, at least 15, at least 20, at least 30, at least 50, at least 75, at least 100, at least 200, at least 300, at least 400, at least 500, at least 600, at least 700, at least 800, at least 900, at least 1,000, or at least 10,000 or more guide RNAs, as described above. The guide RNAs can in the form of a dried pellet or an aqueous solution. The guide RNAs can be at least 20, at least 30, at least 50, at least 75, at least 100, at least 150, at least 180, at least 200, at least 220, at least 240, or at least 260 nucleotides long.

In addition to the instructions, the kits can also include one or more control genomes and or oligonucleotides for use in testing the kit. The subject kit can further include instructions for using the components of the kit to practice the subject methods. The instructions for practicing the subject methods are generally recorded on a suitable recording medium. For example, the instructions can be printed on a substrate, such as paper or plastic, etc. As such, the instructions can be present in the kit as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example is a kit that includes a web address where the instructions can be viewed and/or from which the instructions can be downloaded. As with the instructions, this means for obtaining the instructions is recorded on a suitable substrate.

The various components of the kit(s) can be in separate containers, where the containers can be contained within a single housing, e.g., a box.

In some embodiments, the Cas9-associated guide RNAs are each specific for a different, pre-defined, site in genomic DNA.

In some embodiments, the Cas9-associated guide RNAs are each specific for a different, pre-defined, site in mammalian genomic DNA.

In some embodiments, the Cas9-associated guide RNAs are each specific for a different, pre-defined, site in human genomic DNA.

In some embodiments, the Cas9-associated guide RNAs are each specific for a different, pre-defined, site in microbial genomic DNA.

In some embodiments, the kit comprises one or a plurality of Cas9-associated guide RNA binding to the genome of one pathogen and one or a plurality of Cas9-associated guide RNA binding to the genome of another pathogen.

In some embodiments, the kit further comprises a Cas9 nuclease.

In some embodiments, the Cas9-associated guide RNAs are in solution as a mixture.

In some embodiments, the Cas9-associated guide RNAs are tethered to a substrate in an array. In some embodiments, the kit comprises a DNase inhibitor.

### Uses

The enriched double-stranded nucleic acids can be further subject to sequencing or other quantitation or analysis.

Various embodiments pertaining to enrichment of nucleic acid molecules for sequencing applications as well as other nucleic acid molecule analyses have utility in single molecule sequencing applications and direct digital sequencing methods. In some embodiments, technology using single molecule hybridization with barcoded probes can be used to characterize and/or quantify a genomic region. In general, such technology uses molecular “barcodes” and single molecule imaging to detect and count specific nucleic acid targets in a single reaction without amplification. Typically, each color-coded barcode is attached to a single target-specific probe corresponding to a genomic region of interest. Mixed together with controls, they form a multiplexed CodeSet. In some embodiments, two probes are used to hybridize each individual target nucleic acid. In particular arrangements, a Reporter Probe carries the signal and a Capture Probe allows the complex to be immobilized for data collection. After hybridization, the excess probes are removed, and the immobilized probe/target complexes can be analyzed by a digital analyzer for data collection. Color codes are counted and tabulated for each target molecule (e.g., a genomic region of interest). Suitable digital analyzers include nCounter® Analysis System (NanoString™ Technologies; Seattle, Wash.). Methods and reagents including molecular “barcodes”, and apparatus suitable for NanoString™ technology are further described, for example, in U.S. Appl. Pub. Nos. 2010/0112710, 2010/0047924, 2010/0015607, the entire contents of each are herein incorporated by reference.

Additionally, various embodiments pertaining to enrichment of target nucleic acid molecules have utility in other forms of characterization and/or quantification of nucleic acid molecules are known in the art. For example, characterization of nucleic acid molecules to determine the presence or absence of genomic mutations, DNA variants, quantification of DNA or RNA copy number, and other applications can benefit from selection enrichment of target nucleic acid molecules as provided herein. Examples of some methodologies include, but are not limited to, single molecule sequencing (e.g., single molecule real-time sequencing, nanopore sequencing, high-throughput sequencing or Next Generation Sequencing (NGS), etc.), digital PCR, bridge PCR, emulsion PCR, semiconductor sequencing, among others. One of ordinary skill in the art will recognize other nucleic acid interrogation methods and technology that can be suitably used to interrogate and/or benefit from enriched nucleic acid molecules.

Disclosed further herein are methods and compositions for targeted nucleic acid sequence enrichment for a variety of nucleic acid molecule analyses applications. In particular, some aspects of the present technology are directed to methods and compositions for targeted nucleic acid molecules enrichment and uses of such enrichment for error-corrected nucleic acid sequencing applications that provide improvement in the cost, conversion of molecules sequenced and the time efficiency of generating labeled molecules for targeted ultra-high accuracy sequencing.

In some embodiments, it is advantageous to process nucleic acid molecules so as to improve the efficiency, accuracy, and/or speed of a sequencing process. In accordance with further aspects of the present technology, the efficiency of, for example, duplex-sequencing can be enhanced by targeted nucleic acid fragmentation. Classically, nucleic acid (e.g., genome, mitochondrial, plasmid, etc.) fragmentation is achieved either by physical shearing (e.g., sonication) or relatively nonsequence-specific enzymatic approaches that utilize an enzyme cocktail to cleave DNA phosphodiester bonds. The result of either of the above methods is a sample where the intact nucleic acid molecules (e.g., genomic DNA (gDNA)) is reduced to a mixture of randomly or semi-randomly sized nucleic acid fragments. While effective, these approaches generate variable sized nucleic acid fragments which can result in amplification bias (e.g., short fragments tend to PCR amplify more efficiently than longer fragments and can cluster amplify more easily during polony formation) and uneven depth of sequencing.

In some embodiments, quantitation can be or comprise spectrophotometric analysis, real-time PCR, and/or fluorescence-based quantitation (e.g., using fluorescent dye tagging). In some embodiments, sequencing can be or comprise Sanger sequencing, shotgun sequencing, bridge PCR, nanopore sequencing, single molecule real-time sequencing, ion torrent sequencing, pyrosequencing, digital sequencing (e.g., digital barcode-based sequencing), sequencing by ligation, polony-based sequencing, electrical current-based sequencing (e.g., tunneling currents), sequencing via mass spectroscopy, microfluidics-based sequencing, Illumina Sequencing, next generation sequencing, massively parallel and any combination thereof.

The above-described method can be used to fragment a genome in a defined way, i.e., to produce fragments of one or more chosen regions of a genome. The fragments produced by the subject method can be arbitrarily chosen or, in some embodiments, can have a common function, structure or expression. While the above-described method is not so limited, the method can be employed to isolate promoters, terminators, exons, introns, entire genes, homologous genes, sets of gene sequences that are linked by function, expression or sequence, regions containing insertion, deletion or translocation breakpoints or SNP-containing regions, for example. Alternatively, the method could be used to reduce the sequence complexity of a genome prior to analysis, or to enrich for genomic regions of interest.

In certain embodiments the method can be used to produce fragments of interest (i.e., one or more regions of a genome), where the resultant sample is at least 50% free, e.g., at least 80% free, at least 90% free, at least 95% free, at least 99% free of the other parts of the genome. In particular embodiments, the products of the method can be amplified before analysis. In other embodiments, the products of the method can be analyzed in an unmodified form, i.e., without amplification.

As noted above, the method can be employed to isolate a region of interest from a genome. The isolated region can be analyzed by any analysis method including, but not limited to, DNA sequencing (using Sanger, pyrosequencing or the sequencing systems of Roche/454, Helicos, Illumina/Solexa, and ABI (SOLiD)), a polymerase chain reaction assay, a hybridization assay, a hybridization assay employing a probe complementary to a mutation, a microarray assay, a bead array assay, a primer extension assay, an enzyme mismatch cleavage assay, a branched hybridization assay, a NASBA assay, a molecular beacon assay, a cycling probe assay, a ligase chain reaction assay, an invasive cleavage structure assay, an ARMS assay, or a sandwich hybridization assay, for example. Some products (e.g., single-stranded products) produced by the method can be sequenced and analyzed for the presence of SNPs or other differences relative to a reference sequence. As would be clear to one skilled in the art, the proposed method can be useful in several fields of genetic analysis, by allowing the artisan to focus his or her analysis on a genomic region of interest.

The subject methods find particular use in SNP haplotyping of a chromosomal region that contains two or more SNPs, for enriching for DNA sequences for paired-end sequencing methods, for generating target fragments for long-read sequences, isolating inversion, deletion, and translocation breakpoints, for sequencing entire gene regions (exons and introns) to uncover mutations causing aberrant splicing or regulation, and for the production of long probes for chromosome imaging, e.g., Bionanomatrix, optical mapping, or fiber-FISH-based methods.

In particular cases, the methods described above can also be used for long-range haplotyping by using hemizygous deletions to differentially label maternal and paternal chromosomes. The method can be employed to capture such hemizygous sequences together with adjoining sequence. In this way, maternal and paternal copies of DNA could be separated and analyzed independently. This would enable haplotype phased sequencing.

Although the foregoing disclosure has been described in some detail by way of illustration and example for purposes of clarity of understanding, it is readily apparent to those of ordinary skill in the art in light of the teachings of this disclosure that certain changes and modifications can be made thereto without departing from the spirit or scope of the appended claims.

In some embodiments, Nextera library preparation (available from Illumina, Inc, San Diego, Calif.) is performed to fragment input DNA and introduce sequencing primers, and then the fragmented DNA is contacted with the CRISPR-Cas system provided herein to form a complex. The complex is pulled down and the target DNA can be released from the complex, e.g., using EDTA, heat, SDS, and RNase. The sequencing can then be performed.

In other aspects, the present disclosure provides a method of enriching double-stranded DNA using multiple wild-type Cas9 containing two nuclease domains. In some embodiments, provided herein is a method for enriching a target nucleic acid including: providing a population of Cas9 proteins programmed with a set of crRNAs, wherein the set of crRNAs contains crRNAs complementary to a series of different regions of the target nucleic acid; contacting the target nucleic acid with the population of Cas9 proteins programmed with the set of crRNAs to generate a series of nucleic acid fragments, and ligating adaptors to at least one of nucleic acid fragments, wherein the Cas9 protein retains two nuclease domains.

In some embodiments, the set of crRNAs contains crRNAs complementary to two different regions of the target nucleic acid. The method provided herein can be useful for enriching a long DNA fragment. In some embodiments, the space between the two different region is longer than 10 kb.

In some embodiments, the target nucleic acid is a double-stranded DNA. In some embodiments, the target nucleic acid is a genomic DNA, a chromosomal DNA, a genome, or a partial genome.

Two Cas9 proteins each containing two nuclease domains can be used to treat a double-stranded nucleic acid. Each Cas9 is programmed with a crRNA targeting to a different region on the double-stranded DNA, and thus the reaction generates a double-stranded DNA fragment between the two cutting sites. The DNA fragment can be ligated to adaptors and be prepared for other process and/or analysis, e.g., pull down and sequencing.

In other aspects, the present disclosure provides methods of Cas9 mediated nucleic acid fragmentation and targeted sequencing. The present disclosure provides a method for fragmenting DNA in a sequence specific manner in user defined regions, and generating nucleic acid fragments for subsequent sequencing, e.g., DNA fragments amendable for incorporation into Illumina's sequencing libraries. In some embodiments, the method for sequencing a target nucleic acid provided herein includes providing a population of Cas9 proteins programmed with a set of crRNAs, wherein the set of crRNAs contains crRNAs complementary to a series of different regions across the target nucleic acid; contacting the target nucleic acid with the population of Cas9 proteins programmed with the set of crRNAs to generate a series of nucleic acid fragments and sequencing the series of nucleic acid fragments.

In some embodiments, targeted fragmentation of nucleic acid can be achieved by preparing a population of Cas9 proteins that are programmed with crRNAs targeting regions tiled across the target nucleic acid. In some embodiments, the Cas9 proteins provided herein retain two nuclease domains, they can generate double-stranded nucleic acid breaks and thus a series of nucleic acid fragments. These nucleic acid fragments can be further subjected to nucleic acid sequencing workflows.

In some embodiments, the target nucleic acid molecules provided herein are double-stranded DNA. In some embodiments, the target nucleic acid molecules provided herein are genomic DNA, chromosomal DNA, genomes, or partial genomes.

In some embodiments, the nucleic acid fragments can be amplified, e.g., using limited-cycle polymerase chain reaction (PCR), to introduce other end sequences or adaptors, e.g., index, universal primers and other sequences required for cluster formation and sequencing.

In some embodiments, the sequencing the nucleic acid fragments includes use of one or more of sequencing by synthesis, bridge PCR, chain termination sequencing, sequencing by hybridization, nanopore sequencing, and sequencing by ligation.

In some embodiments, the sequencing methodology used in the method provided herein is sequencing-by-synthesis (SBS). In SBS, extension of a nucleic acid primer along a nucleic acid template (e.g., a target nucleic acid or amplicon thereof) is monitored to determine the sequence of nucleotides in the template. The underlying chemical process can be polymerization (e.g., as catalyzed by a polymerase enzyme). In a particular polymerase-based SBS embodiment, fluorescently labeled nucleotides are added to a primer (thereby extending the primer) in a template dependent fashion such that detection of the order and type of nucleotides added to the primer can be used to determine the sequence of the template.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568 and 6,274,320, each of which is incorporated herein by reference). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system. Excitation radiation sources used for fluorescence-based detection systems are not necessary for pyrosequencing procedures. Useful fluidic systems, detectors and procedures that can be adapted for application of pyrosequencing to amplicons produced according to the present disclosure are described, for example, in PCT/US11/57111, US 2005/0191698 A1, U.S. Pat. Nos. 7,595,883 and 7,244,559, each of which is incorporated herein by reference.

Some embodiments can utilize methods involving the real-time monitoring of DNA polymerase activity. For example, nucleotide incorporations can be detected through fluorescence resonance energy transfer (FRET) interactions between a fluorophore-bearing polymerase and gamma-phosphate-labeled nucleotides, or with zeromode waveguides (ZMWs). Techniques and reagents for FRET-based sequencing are described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008), the disclosures of which are incorporated herein by reference.

Some SBS embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

Another useful sequencing technique is nanopore sequencing (see, for example, Deamer et al. Trends Biotechnol. 18, 147-151 (2000); Deamer et al. Acc. Chem. Res. 35:817-825 (2002); Li et al. Nat. Mater. 2:611-615 (2003), the disclosures of which are incorporated herein by reference). In some nanopore embodiments, the target nucleic acid or individual nucleotides removed from a target nucleic acid pass through a nanopore. As the nucleic acid or nucleotide passes through the nanopore, each nucleotide type can be identified by measuring fluctuations in the electrical conductance of the pore. (U.S. Pat. No. 7,001,792; Soni et al. Clin. Chem. 53, 1996-2001 (2007); Healy, Nanomed. 2, 459-481 (2007); Cockroft et al. J. Am. Chem. Soc. 130, 818-820 (2008), the disclosures of which are incorporated herein by reference).

Additional details of Cas protein/gRNA known in the art are provided in U.S. Pat. No. 9,873,907, CA2955382, WO2019/178577, WO2016/028887, WO2019/030306, US2019/0382824, WO2017/197027, US2015/0211058, US2014/0356867, WO2015/075056, and US2020/0024654, of which each is incorporated by reference herein in its entirety.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The following examples are provided for the purpose of illustration and are not intended to limit the scope of the present disclosure.

### EXAMPLES

### Example 1

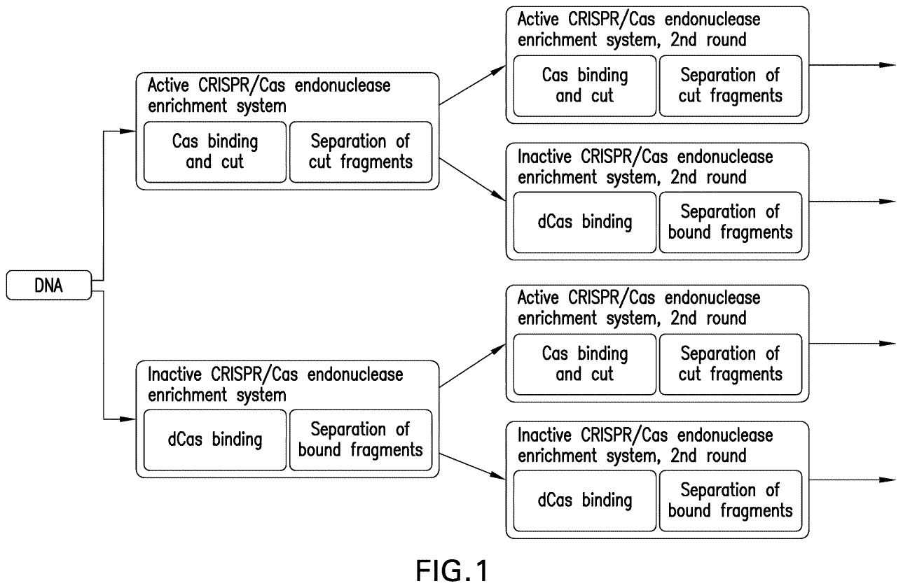

In one set of workflows, a combination of active and/or inactive CRISPR/Cas enrichment systems are used sequentially with a separation step in between (FIG. 1).

### Example 1-1

DNA can be digested by a first (or first set of) Cas protein/gRNA complex, which is specific to a first (or first set of) locus in the target region. After the first digestion, a first separation is used to enrich target DNA population and reduce nontarget DNA representation. The first separated DNA containing higher level of target DNA, is subject to another digestion by a second (or second set of) Cas protein/gRNA complex, which is specific to a second (second set of) locus in the target region. The first (first set of) target locus can be different from the second (or second set of) locus to increase selectivity. After second digestion, the second separation is used to further enrich target DNA. When multiple target regions are enriched together, a set of Cas protein/gRNA complexes are used together in each step.

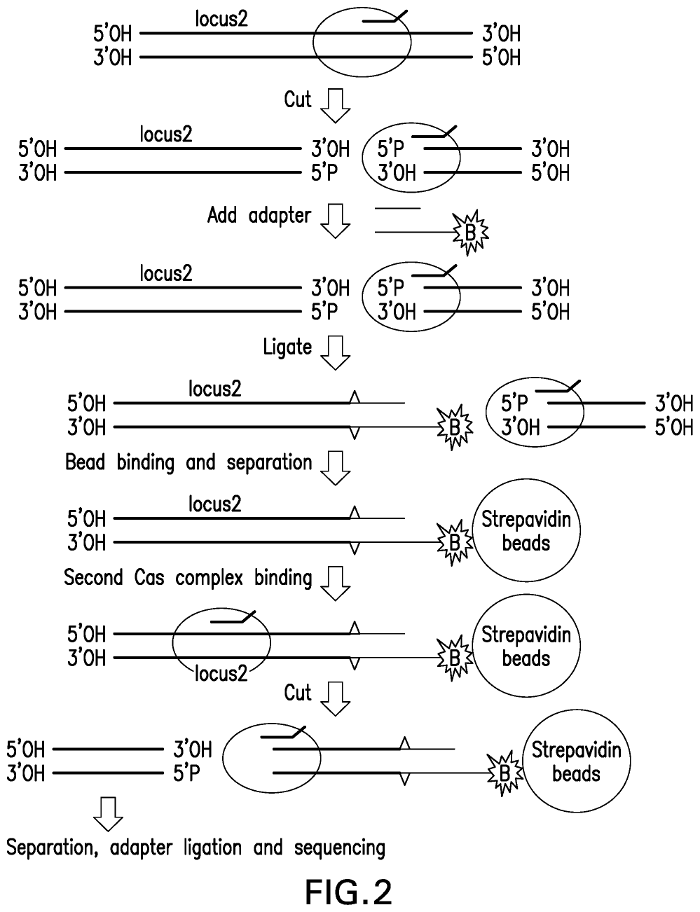

In one example (FIG. 2), two gRNA are designed, both targeting the same target region at different sites. The sample DNA is first dephosphorylated to block existing ends from next ligation. The sample DNA is cut by the first Cas protein/gRNA complex. Then a biotin labeled adapter oligo is ligated to the freshly generated dsDNA cut ends from Cas protein action (and to a small amount of random breaks generated during handling). Next, streptavidin bead is used to separate the adapter ligated DNA from the rest. With the adapter ligated DNA bound on the beads, a second specific Cas protein/gRNA complex is added to cut DNA at the second locus. This time the cut DNA will be released from the beads and can be collected for next adapter ligation and sequencing. The first Cas protein cut, adapter ligation and bead separation remove nontarget DNA substantially, although not completely. If the resulting adapter ligated DNA were to be sequenced directly, it would have a low enrichment factor. The second Cas protein/gRNA cut, while on the beads, will add further specificity to the system, because nontarget DNA evading the first set of steps would not be cut and released to the solution after the second cut.

The first separation can be achieved by other methods mentioned previously, for example by affinity labels on the Cas protein, gRNA, or target DNA. The initial blocking of accessible ends can also be achieved by other means mentioned previously, e.g., by blocking oligo ligation, hairpin oligo ligation, or blocking nucleotide addition.

### Example 1-2

DNA can be digested by a first (or first set of) Cas protein/gRNA complex, which is specific to a first (or first set of) locus in the target region. After the first digestion, a first separation is used to enrich target DNA population and reduce nontarget DNA representation. The first separated DNA containing higher level of target DNA, is subject to another binding by a second (or second set of) inactive dCas protein/gRNA complex, which is specific to a second (or second set of) locus in the target region. The first (or first set of) target locus can be different from the second (or second set of) locus to increase selectivity. After the second binding, the second separation is used to further enrich target DNA. When multiple target regions are enriched together, a set of Cas protein/gRNA complexes are used together in each step.

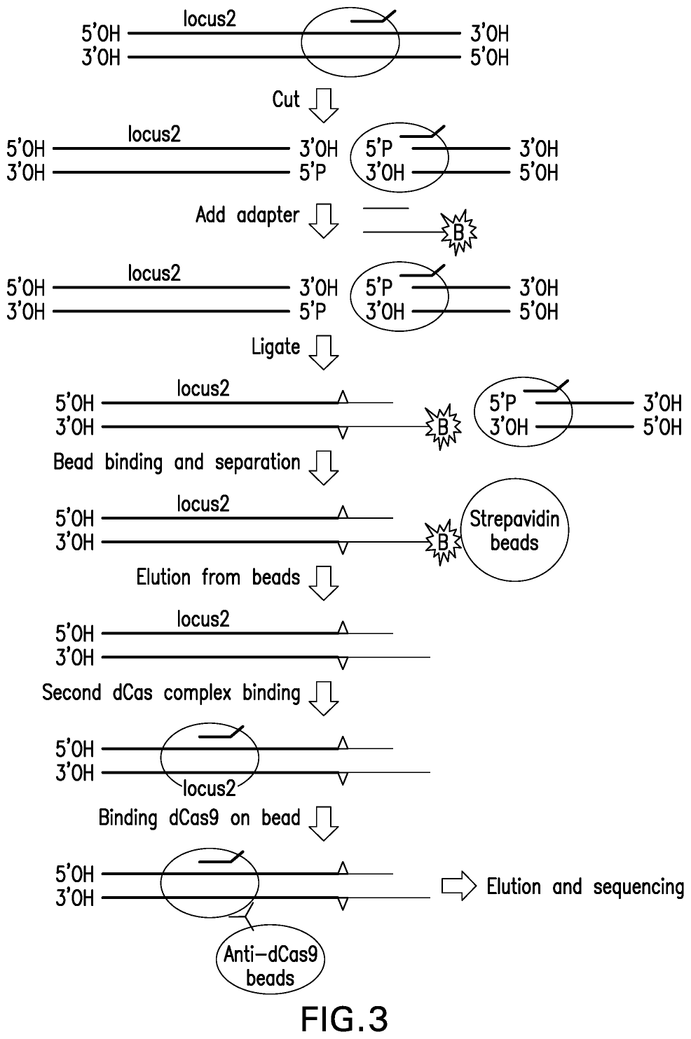

In one example (FIG. 3), two gRNA are designed, both targeting the same target region at different sites. The sample DNA is first dephosphorylated to block existing ends from next ligation. The sample DNA is cut by the first Cas protein/gRNA complex. Then a biotin labeled adapter oligo is ligated to the freshly generated dsDNA cut ends from Cas protein action (and to a small amount of random breaks generated during handling). Next, streptavidin bead is used to separate the adapter ligated DNA from the rest. After beads wash, the adapter ligated DNA is eluted from the beads. A second dCas protein/gRNA complex is provided to bind to the second locus in the adapter ligated DNA. dCas protein bound target DNA can be separated from remaining nontarget DNA through affinity binding, e.g. beads carrying antibodies against dCas protein. The first Cas protein cut, adapter ligation and bead separation remove nontarget DNA substantially, although not completely. If the resulting adapter ligated DNA were to be sequenced directly, it would have a low enrichment factor. The second dCas protein/gRNA binding will add further specificity to the system, because nontarget DNA evading the first set of steps would not be bound by the second specific dCas protein/gRNA complex.

The first separation can be achieved by other methods mentioned previously, for example by affinity labels on the Cas protein, gRNA, or target DNA. The initial blocking of accessible ends can also be achieved by other means mentioned previously, e.g., by blocking oligo ligation, hairpin oligo ligation, or blocking nucleotide addition.

### Example 1-3

DNA can be bound by a first (or first set of) dCas protein/gRNA complex, which is specific to a first (or first set of) locus in the target region. A first separation is used to enrich target DNA population and reduce nontarget DNA representation. The first separated DNA containing higher level of target DNA, is subject to another binding by a second (or second set of) active Cas protein/gRNA complex, which is specific to a second (or second set of) locus in the target region. The first (or first set of) target locus can be different from the second (or second set of) locus to increase selectivity. After cutting by the second (or second set of) Cas protein/gRNA complex, a second separation is used to further enrich target DNA. When multiple target regions are enriched together, a set of Cas protein/gRNA complex are used together in each step.

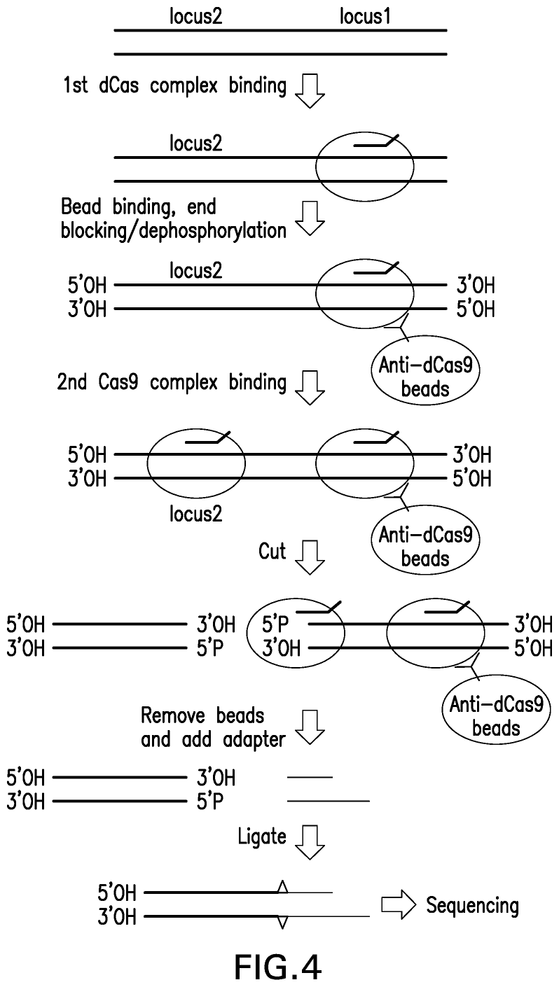

In one example (FIG. 4), two gRNA are designed, both targeting the same target region at different sites. The sample DNA is bound by the first inactive dCas protein/gRNA complex. dCas protein bound target DNA can be separated from remaining nontarget DNA through affinity binding, e.g. beads carrying antibodies against dCas protein. While on beads, bound DNA is dephosphorylated to block existing ends from next ligation. DNA dephosphorylation can also be carried out before dCas protein binding. A second specific and active Cas protein/gRNA complex is added to cut the bead bound DNA at the second locus. The cut DNA will be released from the beads and can be collected for adapter ligation and sequencing. The first dCas protein binding and bead separation removes nontarget DNA substantially, although not completely. If the resulting dCas protein/DNA complex were to be sequenced directly, it would have a low enrichment factor. The second Cas protein/gRNA cut, while on the beads, will add further specificity to the system, because nontarget DNA evading the first set of steps would not be cut and released to the solution after the second Cas protein cut.

The first dCas protein/DNA complex can be separated from other DNA by various affinity methods mentioned previously, for example, by anti-dCas antibodies, or by affinity labels on either dCas protein or gRNA. The blocking of accessible DNA ends can also be achieved by other means mentioned previously, e.g. by blocking oligo ligation, hairpin oligo ligation, or blocking nucleotide addition.

### Example 1-4

DNA can be bound by a first (or first set of) dCas protein/gRNA complex, which is specific to a first (or first set of) locus in the target region. A first separation is used to enrich target DNA population and reduce nontarget DNA representation. The first separated DNA containing higher level of target DNA, is subject to another binding by a second (or second set of) dCas protein/gRNA complex, which is specific to a second (or second set of) locus in the target region. The first (or first set of) target locus can be different from the second (or second set of) locus to increase selectivity. After the second dCas protein/gRNA binding, a second separation is used to further enrich target DNA. When multiple target regions are enriched together, a set of Cas protein/gRNA complex are used together in each step.

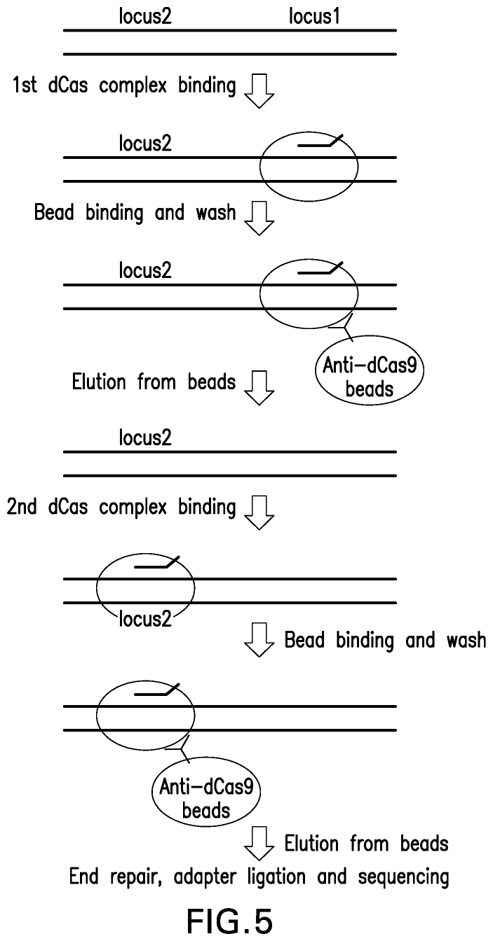

In one example (FIG. 5), two gRNA are designed, both targeting the same target region at different sites. The sample DNA is bound by the first inactive dCas protein/gRNA complex. dCas protein bound target DNA can be separated from nontarget DNA through affinity binding, e.g., beads carrying antibodies against dCas protein. After bead wash, DNA is eluted off the bead. A second dCas protein/gRNA complex is provided to bind to the second locus in the eluted DNA. dCas protein bound target DNA can be separated from remaining nontarget DNA through affinity binding, e.g., beads carrying antibodies against dCas protein. The first dCas protein binding and bead separation removes nontarget DNA substantially, although not completely. If the resulting DNA were to be sequenced directly, it would have a low enrichment factor. The second dCas protein/gRNA binding will add further specificity to the system, because nontarget DNA evading the first set of steps would not be bound by the second specific dCas protein/gRNA complex.

The first and second dCas protein/DNA complex can be separated from other DNA by various affinity methods mentioned previously, for example, by anti-dCas antibodies, or by affinity labels on either dCas protein or gRNA.

### Example 2

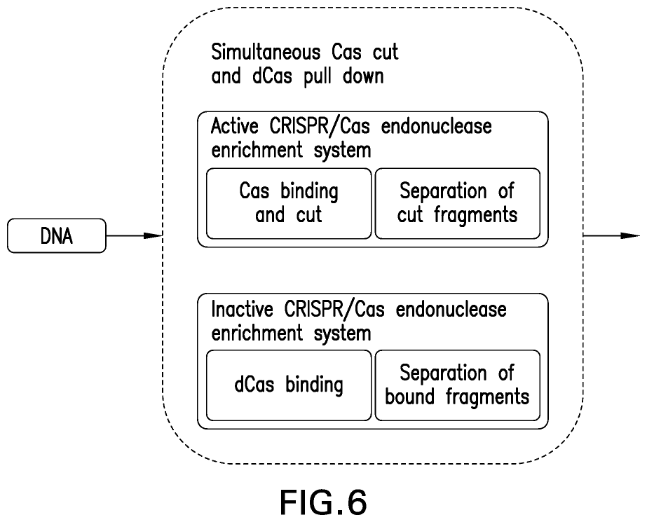

In another set of workflows, a combination of active and/or inactive CRISPR/Cas enrichment systems are applied together in the same reaction tube (FIG. 6). Most of the previously described separation methods can be used. Combinations of separation methods need to be chosen carefully to make them compatible in the same Cas enzyme reaction.

In one example (FIG. 7), two gRNA are designed, both targeting the same target region at different sites. The sample DNA is first dephosphorylated to block existing ends from next ligation. An active Cas protein/gRNA complex targeting locus 1 and an inactive dCas protein/gRNA complex targeting locus 2 are provided to the treated DNA at the same time. The gRNA for dCas protein and locus 2 is also biotinylated. Both active and inactive Cas/gRNA complex now bind to DNA at respective sites. Active Cas protein/gRNA complex at locus 1 will cut target DNA to generate fresh DNA cut ends. The adapter oligo is ligated to the freshly generated dsDNA ends from Cas protein action (and to a small amount of random breaks generated during handling). Afterwards, streptavidin bead is provided to bind biotin on the second dCas protein/gRNA complex associated with locus 2, and to separate target DNA carrying locus 2 from the rest of DNA. Target DNA can now be eluted from the beads and ready for sequencing.

The first Cas protein cut and adapter ligation “virtually separate” target DNA from nontarget DNA substantially, although not completely. If the resulting adapter ligated DNA were to be sequenced directly, it would have a low enrichment factor. The second separation using dCas protein/gRNA complex bound on the second locus will add further specificity to the system, because nontarget DNA accidentally ligated to the adapter would not be bound by second dCas protein/gRNA complex, and nontarget DNA accidentally bound by second dCas protein/gRNA complex would not be ligated to the adapter at the first cut locus.

The first “virtual separation” can be ligating an adapter to either blunt ends (e.g. generated by Cas9) or sticky ends (e.g. generated by Cas12a) at the cut site, in combination with or without pre-treating DNA to block native fragment ends from ligation. The first and second sites and their association with either active Cas or dCas protein/gRNA complex can also be reversed to what is illustrated in FIG. 7.

When multiple target regions are enriched together, two sets of Cas protein/gRNA complex, one for active Cas protein and one for inactive dCas protein, can be used together.

### Example 3

For a method using a combination of active and inactive CRISPR/Cas enrichment systems in the same reaction tube (FIG. 7), 6 sgRNAs were designed to target human ribosomal gene for 28S rRNA. Four sgRNAs were assembled with inactive Cas9 (dCas9) for binding enrichment (Bind 1, 2, 3, 4). The other two were assembled with active Cas9 for cutting enrichment (Cut 1, 2). The underlined is the target specific sequence. The rest is the scaffold sequence. See Table 1.

TABLE 1

| SEQ | | |
|---|---|---|
| ID | | |
| NO: | Name | Sequence |
| 1 | Cut | CTCGACTGCCGGCGACGGCCGTTTAAGAGCTATGCTGGAA |
| | 1 | CAGCATAGCAAGTTTAAATAAGGCTAGTCCGTTATCAACTT |
| | | GAAAAAGTGGCACCGAGTCGGTGCTTTTTTT |
| 2 | Cut | GAGGCCATCGCCCGTCCCTTGTTTAAGAGCTATGCTGGAAA |
| | 2 | CAGCATAGCAAGTTTAAATAAGGCTAGTCCGTTATCAACTT |
| | | GAAAAAGTGGCACCGAGTCGGTGCTTTTTTT |
| 3 | Bind | GCTTTTTGATCCTTCGATGTGTTTAAGAGCTATGCTGGAAA |
| | 1 | CAGCATAGCAAGTTTAAATAAGGCTAGTCCGTTATCAACTT |
| | | GAAAAAGTGGCACCGAGTCGGTGCTTTTTTT |
| 4 | Bind | TCCGCACCGGACCCCGGTCCGTTTAAGAGCTATGCTGGAA |
| | 2 | ACAGCATAGCAAGTTTAAATAAGGCTAGTCCGTTATCAAC |
| | | TTGAAAAAGTGGCACCGAGTCGGTGCTTTTTTT |
| 5 | Bind | GGAGCCCGCCCCCTCCGGGGGTTTAAGAGCTATGCTGGAA |
| | 3 | CAGCATAGCAAGTTTAAATAAGGCTAGTCCGTTATCAACTT |
| | | GAAAAAGTGGCACCGAGTCGGTGCTTTTTTT |
| 6 | Bind | AACCAGGATTCCCTCAGTAAGTTTAAGAGCTATGCTGGAA |
| | 4 | ACAGCATAGCAAGTTTAAATAAGGCTAGTCCGTTATCAAC |
| | | TTGAAAAAGTGGCACCGAGTCGGTGCTTTTTTT |

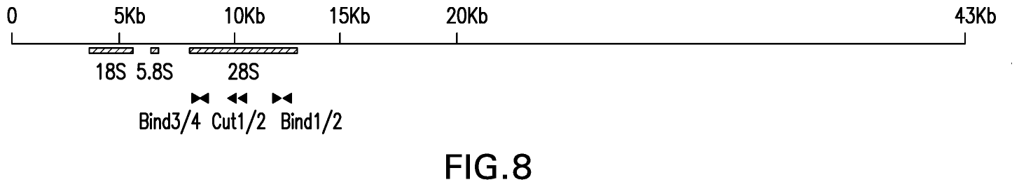

The human ribosomal DNA sequences are clustered in a 43 Kb repeating unit across several chromosomes. The target position and direction of these sgRNAs in relating to the repeating unit is shown in FIG. 8.

The copy number of this 43 Kb repeating unit is polymorphic across human population. For this experiment, we used NA12878 human genomic DNA purchased from Coriell Institute. It was a fully characterized human genomic DNA for which the whole genome sequencing results were available to the public. The copy number of 28S rDNA was estimated to be 57 per copy of NA12878 genome.

Three enrichment methods were used to enrich target region: binding workflow, cutting workflow, and combined workflow.

For binding workflow, 5 ug NA12878 gDNA was mixed with 2.5 pmol of each dCas9 RNP (Bind 1, 2, 3, 4) in 40 ul of 1×NEB CutSmart buffer. The reaction was incubated at 37° C. for 20 min allowing dCas9 RNPs to bind to the target region. The dCas9 was biotin-labeled inactive Cas9 protein from SIGMA-ALDRICH. After binding, 50 uL of Dynabeads-streptavidin Cl beads from Thermo Fisher Scientific was added to the solution to immobilize the dCas9 RNPs along with bound DNA fragments. The beads were washed three times with BWB buffer (5 mM Tris-HCl pH 7.5, 1 M NaCl). After washing, 30 uL elution buffer (EB) was added to the beads and heated to 85° C. for 5 min to elute the DNA fragments. The eluted DNA was then used for Nanopore library preparation following manufacturer's manual.

For cutting workflow, the free ends of 5 ug NA12878 gDNA were first blocked by dephosphorylation using NEB Quick CIP enzyme. The treated DNA was then mixed with 5 pmol of each Cas9 RNP (Cut 1, 2), 2.4 mM dATP and 5 U of NEB Klenow Fragment (3′->5′ exo-) at 37° C. 20 min for Cas9 cleavage and dA-tailing. The reaction was then heated to 75° C. for 5 min to inactivate the enzymes. The freshly cut DNA was then ligated to Nanopore Adapter for sequencing.

The combined workflow was done as depicted in FIG. 7 with minor modification. Briefly, the free ends of 5 ug NA12878 gDNA were first blocked by dephosphorylation using NEB Quick CIP enzyme. The treated DNA was then mixed with 5 pmol of each Cas9 RNP (Cut 1, 2), 2.5 pmol of each dCas9 RNP (Bind 1, 2, 3, 4), 2.4 mM dATP and 5 U of NEB Klenow Fragment (3′->5′ exo-) at 37° C. for 30 min for dCas9 binding, Cas9 cleavage, and dA-tailing. The freshly cut DNA was then ligated to Nanopore native barcode sequence. After that, 50 uL of Dynabeads-streptavidin Cl beads from Thermo Fisher Scientific was added to the solution to immobilize the dCas9 RNPs along with bound DNA fragments. The beads were washed three times with BWB buffer (5 mM Tris-HCl pH 7.5, 1 M NaCl). Afterwards, 65 uL EB was added to beads, and heated to 8° 5 C for 5 min to elute barcode-ligated DNA fragments. The barcoded DNA was then ligated to Nanopore Adapter for sequencing.

The Nanopore sequencing results are summarized in Table 2.

TABLE 2

| Workflow | Binding | Cutting | Combined |
|---|---|---|---|
| sgRNA | Bind 1/2/3/4 | Cut % | Bind 1/2/3/4, |
| | | | Cut1/2 |
| Reads aligned to human genome | 71762 | 96134 | 1504 |
| Reads aligned to target region | 236 | 800 | 95 |
| On target % | 0.33% | 0.83% | 6.32% |
| Enrichment Factor (EF) | 8.6 | 21.9 | 166.2 |

On target % was calculated as:

$$\frac{number of reads aligned to target region}{number of reads aligned to human genome} \times 100 \%$$

where the target region was defined as the first 20 Kb region of the human ribosomal DNA repeating unit.

Enrichment Factor (EF) was calculated as:

$$\frac{On target \% \times Genome size}{Target size \times Target copy number}$$

where:
Genome size=3×10⁹ bp
Target size=2×10⁴ bp
Target copy number=57

When binding or cutting was used separately, the observed EF was 8.6 and 21.9, respectively. By combining the Bind and Cut RNPs into one workflow, we were able to achieve 166.2 EF. This EF was roughly equal to the product of EF (binding) and EF (cutting) (8.6×21.9=188.3), showing synergic effect of the combined enrichment.

### Example 4

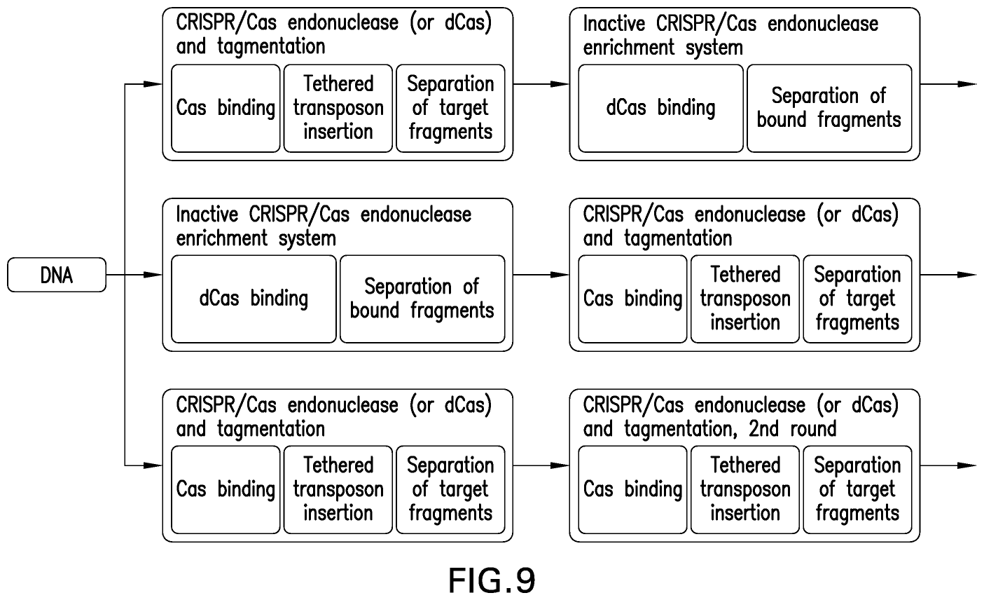

In another set of workflows, a combination of Cas (or dCas)-transposase fusion and/or dCas protein enrichment systems are used sequentially with a separation step in between (FIG. 9). Most of the previously described separation methods can be used. In a two-round enrichment workflow, the Cas tethered tagmentation can be used in the first, second or both rounds.

### Example 4-1

If CRISPR/Cas (or dCas) tethered tagmentation is only used in the first round, the second round of enrichment can be done with catalytically inactive CRISPR/Cas endonuclease enrichment system. In the first round, transposase can be tethered to Cas protein/gRNA complex by any methods mentioned previously. When Cas protein/gRNA binds to target region, tethered transposases will insert a transposon end sequence tag near the binding site. The transposon end sequence tag can also serve as an affinity label to separate target DNA from nontarget DNA, e.g. through additional capture probe binding to the transposon end sequence tag. After the elution of the captured DNA, a second round of enrichment can be done with a second dCas protein/gRNA complex. The target binding site (or sites) in first round can be different from the target binding site (or sites) in second round to increase selectivity. The enriched product after second dCas protein/gRNA binding can be separated by any methods mentioned previously, e.g., by affinity labels on the dCas protein/gRNA complex, or by amplification though CRISPR/Cas mediated sequence specific DNA melting and primer invasion. The enriched material can then be further processed for sequencing. When multiple target regions are enriched together, a set of Cas protein/gRNA complex can be used together in each step.

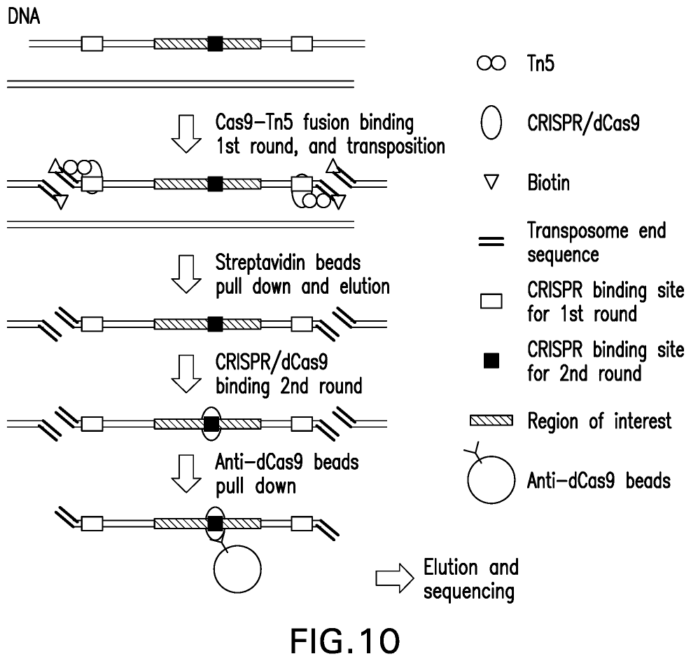

An example of this workflow is illustrated in FIG. 10. The DNA is first enriched by dCas9-Tn5 fusion protein. In this example, first round dCas9/gRNA-Tn5 binding sites are located both upstream and downstream of the target region. The tethered Tn5 inserts transposon end sequences near the binding sites. The transposon end oligos are labelled with biotin tag. Streptavidin coated magnetic bead is used to pull down tagmented DNA. Following wash, DNA is eluted off the beads. New dCas9 protein/gRNA is provided to bind target DNA, at a different site inside the target region. Anti-dCas9 bead is used to capture the target DNA. The bound DNA can be eluted off the bead and ready for further sequencing preparation.

### Example 4-2

If CRISPR/Cas (or dCas) tethered tagmentation is only used in the second round, the first round of enrichment can be done with catalytically inactive CRISPR/Cas endonuclease enrichment system. The enriched product in the first round can be separated any methods mentioned previously, e.g., by affinity labels on the dCas protein/gRNA complex, or by amplification though CRISPR/Cas mediated sequence specific DNA melting and primer invasion. After that, a second round of enrichment can be done with CRISPR/Cas (or dCas) tethered tagmentation. The transposase can be tethered to Cas protein/gRNA complex by any methods mentioned previously. When Cas protein/RNA binds to target region, tethered transposases will insert a transposon end sequence tag near the binding site. The transposon end sequence tag can be a part of sequencing adapter so only target DNA will be sequenced. The transposon end sequence tag can also serve as an affinity label to separate target DNA from nontarget DNA, e.g., through additional capture probe binding to the transposon end sequence tag. The target binding site (or sites) in first round can be different from the target binding site (or sites) in second round to increase selectivity. The enriched material can then be further processed for sequencing. When multiple target regions are enriched together, a set of Cas protein/gRNA complex can be used together in each step.

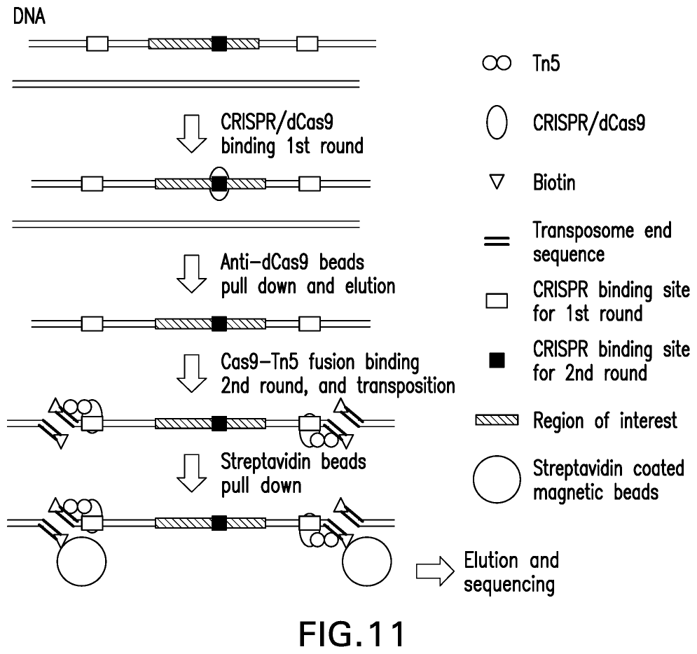

An example of this workflow is provided in FIG. 11. The DNA is first enriched by catalytically inactive CRISPR/Cas endonuclease enrichment system. The dCas9 protein/gRNA binding site is inside the target region. Anti-dCas9 bead is used to separate bound DNA from nontarget DNA. Following bead wash, DNA is eluted off the beads. New dCas9/gRNA-Tn5 complex is added to the eluted DNA. This time the dCas9/gRNA-Tn5 binding sites are located both upstream and downstream of the target region. And tethered Tn5 will insert transposon end sequence near the binding site. The transposon end sequences are labelled with biotin tag. Streptavidin coated magnetic beads are used to pull down tagmented target DNA fragments. The tagmented DNA can be eluted off the bead and ready for further sequencing preparation.

### Example 4-3

The CRISPR/Cas tethered tagmentation can be used in both the 1^{st} and the 2^{nd} round of enrichment. Transposase can be tethered to Cas protein/gRNA complex by any methods mentioned previously. When Cas protein/RNA binds to the target region, tethered transposases will insert a transposon end sequence tag near the binding site. The target binding site (or sites) in first round can be different from the target binding site (or sites) in second round to increase selectivity. The target binding sites could be at the opposite end of the target region, e.g. one at the upstream and the other at the downstream end. After each round of binding and insertion, target DNA need to be separated from nontarget DNA, either through sequencing adapter selection, or capture probe binding. When multiple target regions are enriched together, a set of Cas protein/gRNA complex can be used together in each step.

An example of this workflow is provided in FIG. 11. The DNA is first enriched by a first dCas9/gRNA-Tn5 complex, specific to a binding sites located upstream of the target region. The tethered Tn5 inserts transposon end sequences near the first binding site. The transposon end oligos are labelled with biotin tag. Streptavidin coated magnetic beads are used to pull down tagmented target DNA. Following bead wash, bound DNA is eluted off the bead. A second dCas9/gRNA-Tn5 complex is added, which is specific to a second binding site located downstream of the target region. Tethered Tn5 will insert transposon end sequence near the second binding site. The transposon end oligos are labelled with biotin tag. Streptavidin coated magnetic beads are used again to pull down tagmented target DNA. The tagmented DNA can be eluted off the bead and ready for further sequencing preparation.

The 1^{st} round Cas-tethered transposition and bead separation removes nontarget DNA substantially, although not completely. If the resulting DNA were to be sequenced directly, it would have a low enrichment factor. The 2^{nd} round enrichment will add further specificity to the system, because nontarget DNA evading the first set of steps would not be bound by the second specific dCas protein/gRNA complex. If both rounds of Cas-tethered transposition are added simultaneously without any separation in between, the enrichment factor would be extremely low.

The foregoing description of the specific embodiments will so fully reveal the general nature of the disclosure that others can, by applying knowledge within the skill of the art, readily modify and/or adapt for various applications, without departing from the general concept of the disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be herein incorporated by reference.

1. A method of enriching for target nucleic acid molecules, comprising
(a) binding target nucleic acid molecules in a sample with a first Cas protein/gRNA complex that is specific to a first locus of a target region of the target nucleic acid molecules;
(b) separating the target nucleic acid molecules of (a) from nontarget nucleic acid molecules in the sample; and
(c) binding the separated target nucleic acid molecules of (b) with a second Cas protein/gRNA complex that is specific to a second locus of the target region of the target nucleic acid molecules.

2. (canceled)

3. The method of claim 1, further comprising separating the target nucleic acid molecules of (c) from nontarget nucleic acid molecules.

4. (canceled)

5. The method of claim 1, wherein the first Cas protein/gRNA complex comprises an active Cas protein and the second Cas protein/gRNA complex comprises an active Cas protein, wherein the first Cas protein/gRNA complex comprises an active Cas protein and the second Cas protein/gRNA complex comprises an inactive Cas protein, wherein the first Cas protein/gRNA complex comprises an inactive Cas protein and the second Cas protein/gRNA complex comprises an active Cas protein; the first Cas protein/gRNA complex comprises an inactive Cas protein and the second Cas protein/gRNA complex comprises an inactive Cas protein, or wherein the first Cas protein/gRNA complex comprises an inactive Cas protein and the second Cas protein/gRNA complex comprises an inactive Cas protein.

6.-8. (canceled)

9. The method of claim 5, wherein the active Cas protein cuts the target nucleic acid molecules.

10. The method of claim 9, further comprising ligating an adapter oligonucleotide to the cut ends of the target nucleic acid molecules.

11. The method of claim 10, wherein the adapter oligonucleotide, the Cas protein, the gRNA, or the target nucleic acid molecules are attached to an affinity label.

12.-13. (canceled)

14. The method of claim 11, wherein the separating is performed by binding the target nucleic acid molecules bound to the affinity label to an affinity label partner and eluting the bound target nucleic acid molecules.

15. The method of claim 10, wherein the adapter oligonucleotide ligated to the target nucleic acid molecules cut by the first Cas protein/gRNA complex is attached to an affinity label.

16. The method of claim 15, further comprising eluting the target nucleic acid molecules bound to the affinity label to an affinity label partner before the binding in (c).

17.-18. (canceled)

19. The method of claim 15, wherein the affinity label is an anti-dCas antibody linked to a bead.

20.-23. (canceled)

24. A method of enriching for target nucleic acid molecules, comprising
(a) binding target nucleic acid molecules in a sample with a Cas protein/gRNA complex specific to a first locus of a target region of the target nucleic acid molecules;
(b) binding target nucleic acid molecules in the sample with a Cas protein/gRNA complex specific to a second locus of the target region of the target nucleic acid molecules; and
(c) separating the target nucleic acid molecules from nontarget nucleic acid molecules in the sample.

25. (canceled)

26. The method of claim 24, wherein the first locus of the target region is bound by a Cas protein/gRNA complex comprising an active Cas protein and the second locus of the target region is bound by a Cas protein/gRNA complex comprising an inactive Cas protein or wherein the first locus of the target region is bound by a Cas protein/gRNA complex comprising an inactive Cas protein and the second locus of the target region is bound by a Cas protein/gRNA complex comprising an active Cas protein.

27.-28. (canceled)

29. The method of claim 26, wherein the active Cas protein/gRNA complex cuts the target nucleic acid molecules.

30. The method of claim 29, further comprising ligating an adapter oligonucleotide to cut ends of the target nucleic acid molecules.

31. The method of claim 30, wherein the adapter oligonucleotide, the Cas protein, the gRNA, or the target nucleic acid molecules are attached to an affinity label.

32.-33. (canceled)

34. The method of claim 31, wherein the separating is performed by binding the target nucleic acid molecules bound to the affinity label to an affinity label partner and eluting the bound target nucleic acid molecules.

35. The method of claim 24, wherein the first Cas protein/gRNA complex comprises a set of active Cas protein/gRNA or inactive Cas protein/gRNA that are specific to a set of first loci of 2 or more different target regions or wherein the second Cas protein/gRNA complex comprises a set of active Cas protein/gRNA or inactive Cas protein/gRNA that are specific to a set of second loci of 2 or more different target regions.

36. (canceled)

37. The method of claim 1, wherein a transposase is tethered to the first or second Cas protein/gRNA complex and the tethered transposase inserts a transposon end sequence tag in or near a binding site of the complex, wherein the transposase is tethered to the first Cas protein/gRNA complex and the tethered transposase inserts a transposon end sequence tag near the binding site and the second Cas protein/gRNA complex comprises an inactive Cas protein, wherein the first Cas protein/gRNA complex comprises an inactive Cas protein and the transposase is tethered to the second Cas protein/gRNA complex and the tethered transposase inserts a transposon end sequence tag near the binding site, or wherein the transposase is tethered to the first Cas protein/gRNA complex and to the second Cas protein/gRNA complex and the tethered transposases insert a transposon end sequence tag near the binding sites.

38.-40. (canceled)

41. The method of claim 37, wherein the transposase tethered to the first or second Cas protein/gRNA complex is a dCas9-Tn5 fusion protein.

42. The method of claim 37, wherein the transposon end sequence tag is attached to an affinity label.

43. The method of claim 42, further comprising pulling down tagmented nucleic acid molecules with an affinity label partner.

44.-46. (canceled)

47. A method of enriching for target nucleic acid molecules, comprising:
(a) binding target nucleic acid molecules in a sample with one or more first target endonucleases that are specific to a first locus of a target region of the target nucleic acid molecules;
(b) separating the target nucleic acid molecules from nontarget nucleic acid molecules in the sample; and
(c) binding the separated target nucleic acid molecules with one or more second target endonucleases that are specific to a second locus of the target region of the target nucleic acid molecules.

48. (canceled)

49. The method of claim 47, further comprising separating the target nucleic acid molecules of (c) from nontarget nucleic acid molecules.

50. The method of claim 47, further comprising binding the separated target nucleic acid molecules with one or more third target endonucleases that are specific to a third locus of the target region and separating the target nucleic acid molecules from nontarget nucleic acid molecules.

51. The method of claim 47, wherein the first target endonucleases and the second target endonucleases target different loci of the target region or wherein the first target endonucleases, the second target endonucleases, and the third target endonucleases target different loci of the target region.

52.-53. (canceled)

54. The method of claim 47, further comprising releasing the target nucleic acid molecules from the first or second target endonucleases.

55. (canceled)

56. The method of claim 47, wherein the one or more target endonucleases comprises Cas9, CPFl, or a derivative thereof.

57.-62. (canceled)

63. The method of claim 47, further comprising ligating an adapter to at least one of the 5′ or 3′ ends of the cut target nucleic acid molecules.

64. The method of claim 47, wherein a transposase is tethered to the first or second target endonuclease and the tethered transposase inserts a transposon end sequence tag in or near the binding site of the complex.

65.-66. (canceled)

67. The method of claim 63, wherein at least one target endonuclease or adapter is attached to an affinity label.

68.-69. (canceled)

70. The method of claim 67, further comprising capturing the target nucleic acid molecules with an affinity label partner.

71.-75. (canceled)
